(19) United States
(12) Patent Application Publication
(10) Pub. No.: US 2018/0011116 A1
(43) Pub. Date: Jan. 11, 2018

(54) Use of Viscoelastic Analysis for Predicting Massive Hemorrhage

(71) Applicant: The Regents of the University of Colorado, a body corporate, Denver, CO (US)

(72) Inventor: Michael P. Chapman, Denver, CO (US)

(21) Appl. No.: 15/664,565

(22) Filed: Jul. 31, 2017

Related U.S. Application Data
(63) Continuation of application No. PCT/US16/16412, filed on Feb. 3, 2016.
(60) Provisional application No. 62/111,553, filed on Feb. 3, 2015.

(51) Int. Cl. G01N 33/86; A61B 5/02

(52) U.S. Cl. CPC G01N 33/86; A61B 5/02042; A61B 2505/05; G01N 2800/50; G01N 2800/22

(57) ABSTRACT

The invention provides methods for identifying a patient as likely to have an onset of massive hemorrhage. In one embodiment, the invention provides a method for identifying a patient as likely to have an onset of massive hemorrhage, the method comprising measuring at least one of first coagulation characteristic parameter reflective of a clotting time in a sample of blood of the patient, a second coagulation characteristic parameter reflective of clot formation in a sample of blood of the patient using the viscoelastic assay to obtain a second result; a third coagulation characteristic parameter reflective of clot strength in a sample of blood of the patient using the viscoelastic assay to obtain a third result; and a fourth coagulation characteristic parameter reflective of clot lysis in a sample of blood of the patient using the viscoelastic assay to obtain a fourth result; wherein, a positive for at least one of the first result, second result, third result and fourth result identifies the patient as likely to have an onset of massive hemorrhage.

### CROSS REFERENCE TO RELATED APPLICATION

This application is a continuation of PCT application no. PCT/US16/16412, filed Feb. 3, 2016, which claims priority to U.S. provisional application No. 62/111,553 filed Feb. 3, 2015, the entire contents of which are hereby incorporated by reference.

### STATEMENT REGARDING FEDERAL SPONSORED RESEARCH OR DEVELOPMENT

This invention was made with government support under Grant Nos. T32-GM008315, P50-GM49222, and UMHL120877 awarded by the National Institutes of Health, and under DoD contract no. W81XWH1220028. The government has certain rights in the invention.

### BACKGROUND

The present invention relates to the fields of medicine and surgery and emergent and chronic critical care for traumatic injury.

Following a traumatic injury (e.g., gunshot wound, car accident, or during surgery), uncontrolled hemorrhage (i.e., uncontrolled bleeding) is a leading cause of early death (Eastridge et al., Journal of Trauma. 71(1 Suppl):S4-8, 2011; Gonzalez et al., Journal of trauma. 62(1):112-9, 2007; Kashuk et al., Journal of trauma 65(2):261-71, 2008). Trauma induced coagulopathy (TIC) is an exacerbating phenomenon observed in many cases of massive hemorrhage, and current practice emphasizes hemostatic resuscitation with blood components (Brohi et al., Current Opinion in Critical Care 13(6): 680-685, 2007; Brohi et al., Annals of Surgery 245(5): 812-818, 2007; Brohi et al., J. of Trauma, 64(5): 1211-1217, 2008; Cohen et al., Annals of Surgery 255(2): 379-385, 2012; Cotton et al., journal of trauma and acute care surgery 73(2):365-70, 2012; Dunn et al., Surg Forum. 30:471-3, 1979). This practice is geared at not only replacing lost oxygen carrying capacity, but in reestablishing normal coagulation and forestalling the “bloody vicious cycle” of hemorrhagic shock, acidosis/hypothermia and coagulopathy. (Kashuk et al., Journal of trauma 22(8):672-9, 1982; Cohen et al., Surg. Clin North Am. 92(4):877-91, 2012). Establishment, by hospital blood banks, of massive transfusion protocols (MTPs) to aid in efficient hemostatic resuscitation of patients with life-threatening hemorrhage, represents an important development in trauma systems management and has been shown to reduce mortality in massively bleeding patients. Triggering an massive transfusion protocol (MTP) activation, however, is not without potentially negative consequences. MTPs consume large amounts of scarce medical resources (e.g. universal donor blood components either transfused or wasted) as well as exposing patients to the risks of transfusion of multiple units of blood products, empirically and potentially needlessly unless patients who truly require an MTP activation are chosen accurately. (Johnson et al., Archives of surgery 145(10):973-7, 2010; Moore et al., Archives of surgery 132(6):620-4, 1997; Watson et al., Journal of trauma. 2009 August; 67(2):221-7; discussion 8-30, 2009; Neal et al., Archives of surgery. 147(6):563-71, 2012).

Thus, there is a need to distinguish patients in massive hemorrhage who truly need a massive transfusion protocol from those patients who do not.

### SUMMARY OF THE EMBODIMENTS

In a first aspect, the invention provides a method for identifying a patient as likely to have an onset of massive hemorrhage. The method comprises measuring at least one of first coagulation characteristic parameter reflective of a clotting time in a sample of blood of the patient, a second coagulation characteristic parameter reflective of clot formation in a sample of blood of the patient using the viscoelastic assay to obtain a second result; a third coagulation characteristic parameter reflective of clot strength in a sample of blood of the patient using the viscoelastic assay to obtain a third result; and a fourth coagulation characteristic parameter reflective of clot lysis in a sample of blood of the patient using the viscoelastic assay to obtain a fourth result; wherein, a positive for at least one of the first result, second result, third result and fourth result identifies the patient as likely to have an onset of massive hemorrhage.

In various embodiments, the first coagulation characteristic parameter is selected from the group consisting of an activated clotting time (ACT) value, a clotting time (CT) value, a reaction time (R) value, and a split point (SP) value. In various embodiments, the first result is positive if the first result is greater than or equal to a value that is equivalent to 152 seconds when the first coagulation characteristic parameter is an ACT value as measured by a thromoboelastography viscoelastic analysis assay.

In various embodiments, the second coagulation characteristic parameter is selected from the group consisting of an α-angle value, a K value, and a CFT value. In various embodiments, the second result is positive if the second result is less than or equal to a value that is equivalent to 61.2 degrees when the second coagulation characteristic parameter is an α-angle value as measured by a thromoboelastography viscoelastic analysis assay.

In various embodiments, the third coagulation characteristic parameter is selected from the group consisting of a maximum amplitude (MA) value, a maximum clot firmness (MCF) value, an A5 value, a A10 value, an A15 value, and an A20 value. In various embodiments, the third result is positive if the third result is less than or equal to a value that is equivalent to 49 mm when the third coagulation characteristic parameter is an MA value as measured by a thromoboelastography viscoelastic analysis assay.

In various embodiments, the fourth coagulation characteristic parameter is selected from the group consisting of an LY30 value and an LI30 value. In various embodiments, the fourth result is positive if the fourth result is greater than or equal to a value that is equivalent to 2.5% when the fourth coagulation characteristic parameter is an LY30 value as measured by a thromoboelastography viscoelastic analysis assay.

In various embodiments, the viscoelastic assay is performed using a thromboelastography analyzer system. In various embodiments, the viscoelastic assay is performed using a thromboelastometry analyzer system.

In various embodiments, the patient is a human patient. In various embodiments, the patient is a trauma patient.

In another aspect, the invention provides a method for identifying a patient as likely to have an onset of massive hemorrhage, comprising: (a) obtaining a normalized first result by: (i) measuring a first coagulation characteristic parameter reflective of a clotting time in a sample of blood of the patient using a viscoelastic assay to obtain a first result, and (ii) dividing the first result by a mean of the first coagulation characteristic parameter in samples of blood of trauma patients using the viscoelastic assay to obtain the normalized first result; (b) obtaining a normalized second result by: (i) measuring a second coagulation characteristic parameter reflective of clot formation in a sample of blood of the patient using the viscoelastic assay to obtain a second result; and (ii) dividing the second result by a mean of the second coagulation characteristic parameter in samples of blood of trauma patients using the viscoelastic assay to obtain the normalized second result; (c) obtaining a normalized third result by: (i) measuring a third coagulation characteristic parameter reflective of clot strength in a sample of blood of the patient using the viscoelastic assay to obtain a third result; and (ii) dividing the third result by a mean of the third coagulation characteristic parameter in samples of blood of trauma patients using the viscoelastic assay to obtain the normalized third result; (d) obtaining a normalized fourth result by: (i) measuring a fourth coagulation characteristic parameter reflective of clot formation speed time in a sample of blood of the patient using the viscoelastic assay to obtain a fourth result; and (ii) dividing the second result by a mean of the fourth coagulation characteristic parameter in samples of blood of trauma patients using the viscoelastic assay to obtain the normalized fourth result; and (e) obtaining a value by subtracting the sum of the normalized third result and the normalized second result from the sum of the normalized first result and the normalized fourth result; wherein a value that is greater than greater than a threshold value established between healthy volunteers and positive control test subjects at a particular site of usage identifies the patient as likely to have an onset of massive hemorrhage. In various embodiments, the patient is a human patient. In various embodiments, the patient is a trauma patient.

In various embodiments, the viscoelastic assay is performed using a thromboelastography analyzer system. In various embodiments, the viscoelastic assay is performed using a thromboelastometry analyzer system.

In various embodiments, the first coagulation characteristic parameter is selected from the group consisting of an activated clotting time (ACT) value, a clotting time (CT) value, a reaction time (R) value, and a split point (SP) value. In various embodiments, the second coagulation characteristic parameter is selected from the group consisting of an α-angle value, a K value, and a CFT value. In various embodiments, the third coagulation characteristic parameter is selected from the group consisting of a maximum amplitude (MA) value, a maximum clot firmness (MCF) value, an A5 value, a A10 value, an A15 value, and an A20 value. In various embodiments, the fourth coagulation characteristic parameter is selected from the group consisting of an LY30 value and an LI30 value.

In yet another aspect, the invention provides a method for identifying a patient as likely to have an onset of massive hemorrhage, comprising: (a) obtaining a normalized first result by: (i) measuring a first coagulation characteristic parameter reflective of clot formation in a sample of blood of the patient using the viscoelastic assay to obtain a first result; and (ii) dividing the second result by a mean of the first coagulation characteristic parameter in samples of blood of trauma patients using the viscoelastic assay to obtain the normalized first result; (b) obtaining a normalized second result by: (i) measuring a second coagulation characteristic parameter reflective of clot strength in a sample of blood of the patient using the viscoelastic assay to obtain a second result; and (ii) dividing the second result by a mean of the second coagulation characteristic parameter in samples of blood of trauma patients using the viscoelastic assay to obtain the normalized second result; and (c) adding the normalized first result to the normalized second result to obtain a sum, where a sum that is greater than greater than a threshold value established between healthy volunteers and positive control test subjects at a particular site of usage identifies the patient as likely to have an onset of massive hemorrhage.

In various embodiments, the viscoelastic assay is performed using a thromboelastography analyzer system. In various embodiments, the viscoelastic assay is performed using a thromboelastometry analyzer system.

In various embodiments, the first coagulation characteristic parameter is selected from the group consisting of an α-angle value, a K value, and a CFT value. In various embodiments, the second coagulation characteristic parameter is selected from the group consisting of a maximum amplitude (MA) value, a maximum clot firmness (MCF) value, an A5 value, a A10 value, an A15 value, and an A20 value.

In various embodiments, the patient is a human patient. In various embodiments, the patient is a trauma patient.

### BRIEF DESCRIPTION OF THE DRAWINGS

The foregoing features of embodiments will be more readily understood by reference to the following detailed description, taken with reference to the accompanying drawings, in which:

FIG. 1 is a schematic drawing showing a typical thromboelastography (TEG) tracing from a healthy person, with the coagulation characteristic parameters depicted on the tracing. As typically presented, time from assay initiation is the dependent variable (on the x-axis) and the amplitude of coupling of the motion of the reaction cup to a centrally suspended pin (a proxy for viscoelastic clot strength) is the readout variable, on the y-axis, reported in FIG. 1 in units of millimeters.

FIG. 2 is a schematic drawing showing a typical thromboelastometry (TEM) tracing from a healthy person, with the coagulation characteristic parameters depicted on the tracing. As typically presented, time from assay initiation is the dependent variable (on the x-axis) clot firmness is on the y-axis.

FIG. 3 is a schematic drawing showing a side-by-side comparison of a typical TEG tracing (top half of figure) and a typical TEM tracing (bottom half of figure). As is clear from FIG. 3, the R value in a TEG tracing is equivalent to a CT value in a TEM tracing, the K value in a TEG tracing is equivalent to a CFT value in a TEM tracing, the MA value in a TEG tracing is equivalent to a MCF value in a TEM tracing.

FIG. 4 is a flow chart showing a non-limiting aspect of the examiner, whereby the results four TEG parameters are obtained, and if any one of the four is positive, the patient is identified as being likely to have an onset of massive hemorrhage. If all four are negative, the patient is cleared and does not need administration of blood (e.g., does not need transfusion of blood such as a transfusion of packed red blood cells).

FIGS. 5A-5D are line graphs showing the receiver operating characteristic (ROC) plots for synthetic TEG parameters for the retrospective (Example I) patients. The parameters shown are activated clotting time (ACT) (FIG. 5A), α-angle (FIG. 5B), maximum amplitude (MA) (FIG. 5C) and LY30 (FIG. 5D). Areas under the ROC curves (AUCs) were: 0.59 (p=0.25) for ACT (FIG. 5A); 0.74 (p=0.0013) for α-angle (FIG. 5B); 0.80 (<0.0001) for MA (FIG. 5C); and 0.67 (p=0.025) for LY30 (FIG. 5D).

FIGS. 6A-6F are line graphs showing the receiver operating characteristic (ROC) for the retrospective (Example I) patients for conventional trauma scoring systems as well as INR and ISS. FIG. 6A shows the results of the INR scoring system, FIG. 6B shows the results for the ABC scoring system, FIG. 6C shows the results for the TASH scoring system, FIG. 6D shows the results for the ISS scoring system, FIG. 6E shows the results for the Resuscitation Outcomes Consortium vital signs standard (ROCVS), and FIG. 6F shows the results for the Denver Health Medical Center Massive Transfusion Protocol (DHMTP) trigger criteria scoring system. AUCs were: 0.59 for INR (p=0.28); 0.50 (p=0.95 for ISS); 0.52 (p=0.81) for ABC; 0.56 (p=0.46) for TASH; 0.55 (p=0.56) for the Resuscitation Outcomes Consortium vital signs standard (ROCVS); and 0.65 (p=0.06′7) for the new Denver Health Medical Center Massive Transfusion Protocol (DHMTP) trigger criteria.

FIGS. 7A-7C are line graphs showing the receiver operating characteristic (ROC) plots for synthetic TEG parameters for the retrospective (Example I) patients. The parameters shown are the global TEG at 90% specificity (FIG. 7A), the four parameter TEG with normalized sum (FIG. 7B), and α-angle plus maximum amplitude (Angle+MA) (FIG. 7C). Areas under the ROC curves (AUCs) were: 0.77 (p=0.0004) for the global TEG parameter (at 90% specificity for each of its components); 0.80 (p<0.00001) for the four-parameter TEG normalized sum (FPNS); and 0.77 (p=0.0003) for the simplified normalized sum of α-angle and MA (Angle+MA).

FIGS. 8A-8D are line graphs showing the receiver operating characteristic (ROC) plots for synthetic TEG parameters applied to prospective test data of Example II. The parameters shown are the global TEG at 90% specificity (FIG. 8A), the four parameter TEG with normalized sum (FIG. 8B), α-angle plus maximum amplitude (Angle+MA) (FIG. 8C), and CR/CFF Global TEG (95% specificity) (FIG. 8D).

FIGS. 9A-9H are line graphs showing the receiver operating characteristic (ROC) plots of various basic TEG parameters applied to prospective test data of Example II. FIG. 9A shows the ROC plot results for the ACT parameter in the CR-TEG assay. FIG. 9B shows the ROC plot results for the α-angle in the CR-TEG assay. FIG. 9C shows the ROC plot results for the maximum amplitude (MA) parameter in the CR-TEG assay. FIG. 9D shows the ROC plot results for the LY30 parameter in the CR-TEG assay. FIG. 9E shows the ROC plot results for the R-time in the CFF-TEG assay. FIG. 9F shows the ROC plot results for the α-angle in the CFF-TEG assay. FIG. 9G shows the ROC plot results for maximum amplitude (MA) parameter in the CFF-TEG assay. FIG. 9H shows the ROC plot results for the LY30 parameter in the CFF-TEG assay. ROC curves for CR-TEG ACT and CFF-TEG R were not significantly different from the line of identity. AUCs for CR-TEG α-angle, MA and LY30 were 0.86, 0.92 and 0.72, respectively. AUCs for CFF-TEG α-angle, MA and LY30 were 0.72, 0.93 and 0.91, respectively.

FIG. 10 is a line graph showing the receiver operating characteristic (ROC) plot for the TCN-TEG LY30 parameter as applied to the prospective test data of Example II. AUC was 0.98 (p<0.0001), with optimized sensitivity of 100.0% and specificity of 94.4% at a threshold of LY30>54.7%.

### DETAILED DESCRIPTION OF SPECIFIC EMBODIMENTS

The invention stems, in part, from the discovery of methods to reliably identify patients who are likely to have massive hemorrhage who need a massive transfusion protocol in order to survive. The publications (including patent publications), web sites, company names, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

The further aspects, advantages, and embodiments of the invention are described in more detail below. The definitions used in this specification and the accompanying claims shall following terms shall have the meanings indicated, unless the context clearly otherwise requires. Any conflict between an art-understood definition of a word or phrase and a definition of the word or phrase as specifically taught in this specification shall be resolved in favor of the latter. As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

When a trauma patient is brought into a treatment center such as an emergency room, uncontrolled hemorrhage can lead to death. However, a recent analysis of data from a three year study of resuscitation strategies of patients (whose inclusion criteria included activation of an MTP) revealed that only 46% of patients, for whom an MTP was activated based upon the judgment of the attending trauma surgeon, actually went on to require a massive transfusion (defined as ≧10 units or packed red blood cells [PRBCs] in 6 hours). It is clear that clinician judgment leaves much to be desired in terms of accuracy of prediction of massive transfusion requirement. Additionally, recent experience with the design of several prospective trials related to trauma resuscitation have highlighted the difficulty in early, prospective selection of patients truly at high risk of major hemorrhage and coagulopathy (Brasel et al., Journal of the American College of Surgeons. 2008 February; 206(2):220-32, 2008; Bulger et al., Archives of surgery. 2008 February; 143(2):139-48; discussion 49); Bulger et al., Annals of surgery. 2011 March; 253(3):431-41; and Raab et al., Critical care medicine. 2008 November; 36(11 Suppl):5474-80)

Viscoelastic hemostatic assays (VHAs) such as thrombelastography (TEG) or rotational thrombelastometry (EM) are coagulation assays suitable for point-of-care use, which are useful for evaluating the various aspects of trauma induced coagulopathy (TIC), including enzymatic phase clotting failure, diminished clot strength and hyperfibrinolysis (Johansson et al., Transfusion. 2013 December; 53(12):3088-99; Kashuk et al., Annals of surgery. 2010 September; 252(3):434-42; Schochl et al., Critical care. 2011; 15(6):R265; Schochl et al., The Journal of trauma. 2009 July; 67(1):125-31). It is difficult to distinguish clinical trauma induced coagulopathy (TIC) and its objective manifestation as uncontrollable hemorrhage and hemorrhage and TIC have a feed-forward, cyclic causal relationship. Nevertheless, as the clinical entities of massive hemorrhage and TIC frequently co-present, massive hemorrhage is a useful proxy metric for coagulopathy and it is logical to utilize early evidence of TIC as a predictor of impending massive hemorrhage.

The invention stems, in part, from the discovery that a single viscoelastic whole blood coagulation assay, performed on blood samples drawn very near to the time of injury, could serve as better predictors of massive hemorrhage than either clinician judgment or typical trauma scoring systems (e.g. ABC and TASH).

As used herein, by “viscoelastic analysis” is meant any analysis method that measures the characteristics of elastic solid (e.g., fibrin solids) and fluids. In other words, viscoelastic analysis allows the study of properties of a viscous fluid, such as blood or a blood sample.

In some embodiments, the viscoelastic analysis is performed under conditions that mimic the conditions in vivo that result in hemostasis. For example, the condition may include a temperature that mimics a body temperature (e.g., a temperature of 37° C.). The condition may also include clot formation and dissolution at flow rates that mimic those found in blood vessels.

In some embodiments, viscoelastic analysis of a blood sample may include subjecting the blood sample to analysis on a hemostasis analyzer instrument. One non-limiting viscoelastic analysis method is the thromboelastography (“TEG”) assay. Thus in some embodiments, the viscoelastic analysis includes subjecting a blood sample to analysis using thromboelastography (TEG), which was first described by Helmut Hartert in Germany in the 1940's.

Various devices that perform thromboelastography, and methods for using it are described in U.S. Pat. Nos. 5,223,227; 6,225,126; 6,537,819; 7,182,913; 6,613,573; 6,787,363; 7,179,652; 7,732,213, 8,008,086; 7,754,489; 7,939,329; 8,076,144; 6,797,419; 6,890,299; 7,524,670; 7,811,792; 20070092405; 20070059840; 8,421,458; US 20120301967; and 7,261,861, the entire disclosures of each of which are hereby expressly incorporated herein by reference.

Thromboelastography (TE) monitors the elastic properties of blood as it is induced to clot under a low shear environment resembling sluggish venous blood flow. The patterns of changes in shear elasticity of the developing clot enable the determination of the kinetics of clot formation, as well as the strength and stability of the formed clot; in short, the mechanical properties of the developing clot. As described above, the kinetics, strength and stability of the clot provides information about the ability of the clot to perform “mechanical work,” i.e., resisting the deforming shear stress of the circulating blood. In essence, the clot is the elementary machine of hemostasis. Hemostasis instruments that measure hemostasis are able to measure the ability of the clot to perform mechanical work throughout its structural development. These hemostasis analyzers measure continuously all phases of patient hemostasis as a net product of whole blood components in a non-isolated, or static fashion from the time of test initiation until initial fibrin formation, through clot rate strengthening and ultimately clot strength through coagulation characteristic.

In some embodiments, the viscoelastic analysis comprises use of a container which is in contact with the blood sample.

As used herein, by “blood” is meant blood or blood components, whether treated (e.g., with citrate) or not treated. Thus, the term “blood” includes, without limitation, whole blood, citrated whole blood, platelets, plasma, fresh frozen plasma, red blood cells, etc.

As used herein, by “container” is meant a rigid surface (e.g., a solid surface), a portion of which contacts a portion of a blood sample placed into the container at any point during the viscoelastic analysis. The portion of the container that contact the portion of blood sample may also be referred to as the “interior” of the container. Note that the phase “into the container” does not mean that the container has a bottom surface which is in contact with the portion of the blood sample. Rather, the container can be a ring-shaped structure, where the inside of the ring is the interior of the container, meaning that the inside of the ring is the portion of the ring-shaped container that contacts a portion of the blood sample. A blood sample can flow into the container and be held there, for example, by vacuum pressure or surface tension.

Still additional types of containers that are included in this definition are those present on cartridges and cassettes (e.g., a microfluidic cartridge), where the cartridge or cassette has multiple channels, reservoirs, tunnels, and rings therein. Each of the contiguous channels (comprising, for example, a channel, a reservoir, and a ring) is a container, as the term is used herein. Hence, there may be multiple containers on one cartridge. U.S. Pat. No. 7,261,861 (incorporated herein by reference) describes such a cartridge with multiple channels or containers. Any of the surfaces in any of the channels or tunnels of the cartridge may be an interior of the container if that surface comes into contact with any portion of the blood sample, at any time during the viscoelastic analysis.

One non-limiting hemostasis analyzer instrument is described in U.S. Pat. No. 7,261,861; US Patent Publication No. US US20070092405; and US Patent Publication No. US20070059840.

Another non-limiting hemostasis analyzer instrument that performs viscoelastic analysis using thromboelastography (TEG) is the TEG thromboelastograph hemostasis analyzer system sold commercially by Haemonetics, Corp. (Braintree, Mass.).

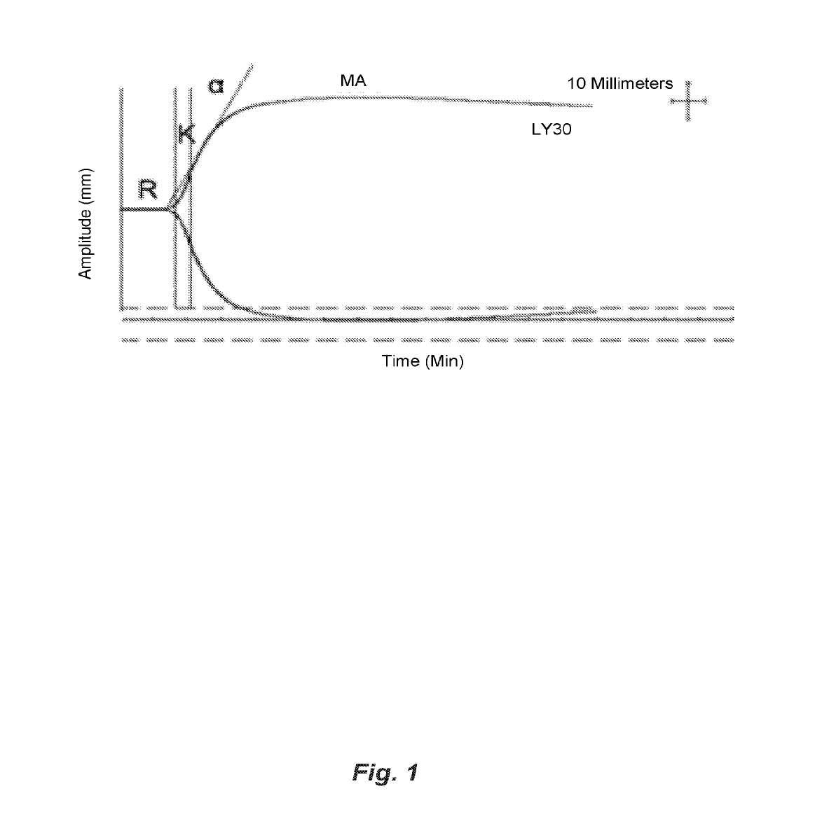

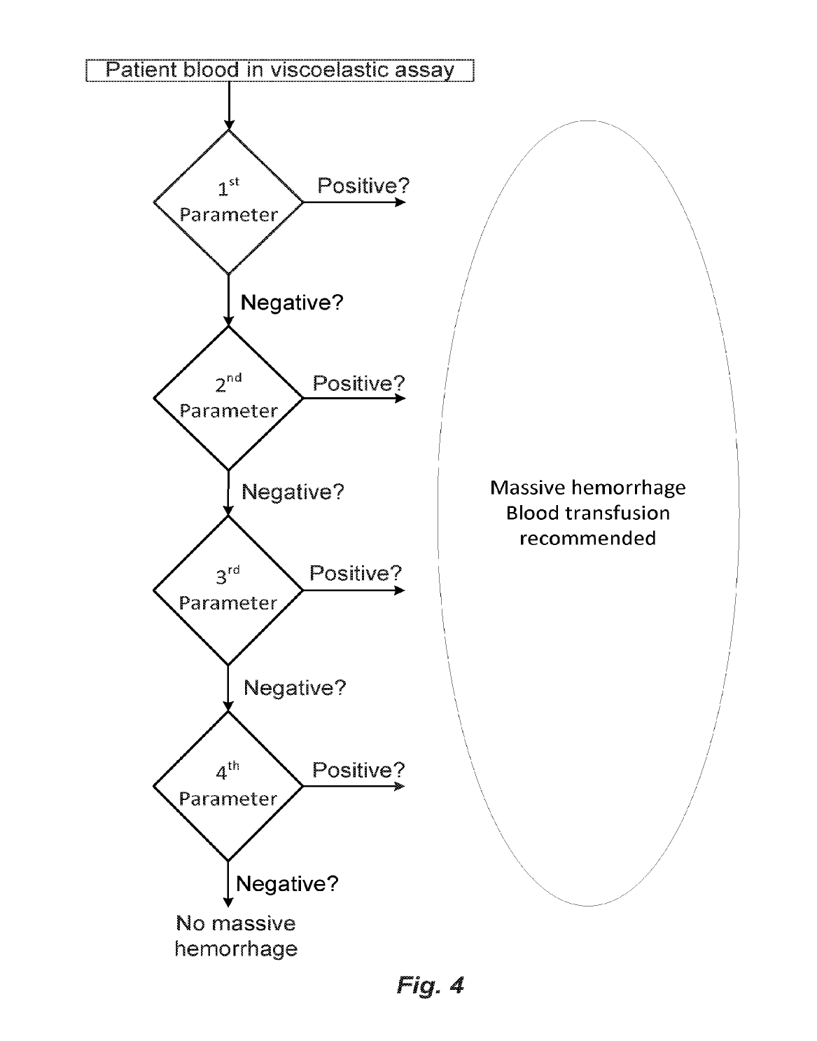

Thus, the TEG assay may be performed using the TEG thromboelastograph hemostasis analyzer system that measures the mechanical strength of an evolving blood cloth. To run the assay, the blood sample is placed into a container (e.g., a cup or a cuvette), and a pin goes into the center of the container. Contact with the interior walls of the container (or addition of a clot activator to the container) initiates clot formation. The TEG thromboelastograph hemostasis analyzer then rotates the container in an oscillating fashion, approximately 4.45 degrees to 4.75 degrees, every 10 seconds, to imitate sluggish venous flow and activate coagulation. As fibrin and platelet aggregates form, they connect the inside of the container with the pin, transferring the energy used to move the container in the pin. A torsion wire connected to the pin measures the strength of the clot over time, with the magnitude of the output directly proportional to the strength of the clot. As the strength of the clot increases over time, a classic TEG tracing curve develops (See FIG. 1). FIG. 1 shows a typical TEG tracing of an untreated blood sample from a healthy individual.

Where there is a pin in the TEG analyzer, the rotational movement of the pin is converted by a transducer to an electrical signal, which can be monitored by a computer including a processor and a control program. The computer is operable on the electrical signal to create a hemostasis profile corresponding to the measured clotting process. Additionally, the computer may include a visual display or be coupled to a printer to provide a visual representation of the hemostasis profile. Such a configuration of the computer is well within the skills of one having ordinary skill in the art. As shown in FIG. 4, the resulting hemostasis profile (i.e., a TEG tracing curve) is a measure of the time it takes for the first fibrin strand to be formed, the kinetics of clot formation, the strength of the clot (measured in millimeters (mm) and converted to shear elasticity units of dyn/cm 2) and dissolution of clot. See also Donahue et al., J. Veterinary Emergency and Critical Care: 15(1): 9-16 (March 2005), herein incorporated by reference

The descriptions for several of these measured parameters are as follows:

R is the period of time of latency from the time that the blood was placed in the thromboelastography analyzer until the initial fibrin formation. In other words, R is the reaction time to clot initiation, as defined as the deflection of the tracing 2 mm from the time axis. R is functionally indistinct from the slight early split point (SP), which is not shown in FIG. 1. R is typically takes about 30 second to about 20 minutes; however the R range will vary based on the particular TEG assay performed (e.g., type of blood sample being tested (e.g., plasma only or whole blood), whether the blood component is citrated or not, etc.). For patients in a hypocoagulable state (i.e., a state of decreased coagulability of blood), the R number is longer, while in a hypercoagulable state (i.e., a state of increased coagulability of blood), the R number is shorter. In the methods described herein, the R value (in minutes or seconds) can be used as a non-limiting coagulation characteristic parameter of clotting time.

The TEG-ACT (i.e., the activated clotting time in a TEG assay) is a calculated parameter based on R, normalized to correspond to traditional (i.e., non-TEG) ACT values. In the methods described herein, the ACT value or TEG-ACT value can be used as a non-limiting coagulation characteristic parameter reflective of clotting time.

K value (measured in minutes) is the clot kinetics parameter. K is the time from the end of R until the clot reaches 20 mm amplitude and this represents the speed of clot formation. This K value is about 0 to about 4 minutes (i.e., after the end of R), or even longer. In some embodiments, the K is never reached because the clot never achieves 20 mm. In a hypocoagulable state, the K number is longer, while in a hypercoagulable state, the K number is shorter. In the methods described herein, the K value can be used as a non-limiting coagulation characteristic parameter reflective of clot formation.

α (alpha) angle measures the rapidity of fibrin build-up and cross-linking (clot strengthening). Thus, the α (alpha) angle is also reflective of clot formation or the coagulation process. The α (alpha) angle is the angle from the baseline to the rising curve's tangent ray, α, drawn from the splitting point of the tracing from baseline, which serves a measurement of clot kinetics. In other words, a (alpha) angle is angle between the line formed from the split point tangent to the curve and the horizontal axis. This angle is typically about 47° to 74° (in a healthy patient). In a hypocoagulable state, the α degree is lower, while in a hypercoagulable state, the α degree is higher. In the methods described herein, the α angle can be used as a non-limiting coagulation characteristic parameter reflective of the rate of clot formation or coagulation.

MA or Maximum Amplitude in mm (millimeters) is maximum amplitude and is reflective of clot strength (see FIG. 1). The MA is a direct function of the maximum dynamic properties of fibrin and platelet bonding and represents the ultimate strength of the blood clot. The MA value is reflective of the coagulation process and is typically from about 54 mm to about 72 mm. The MA occurs typically between about 5 to about 35 minutes after the start of the viscoelastic assay. Note that if the blood sample tested has a reduced platelet function (e.g., platelet-free plasma), this MA represents the strength of the clot based mainly on fibrin. Decreases in MA may reflect a hypocoagulable state (e.g., with platelet dysfunction or thrombocytopenia), whereas an increased MA (e.g., coupled with decreased R) may be suggestive of a hypercoagulable state. In the methods described herein, the MA value can be used as a non-limiting coagulation characteristic parameter reflective of clot strength.

The G-value (not pictured in FIG. 1) is merely a calculated estimate of clot strength based entirely upon MA, but in units of dynes/cm². Thus, the G value is a non-limiting coagulation characteristic parameter reflective of clot strength.

LY30 is a percentage decrease in amplitude 30 minutes after MA and is reflective of clot retraction, or clot lysis. LY30 is thus a percentage of clot lysis (generally from enzymatic degradation of fibrin, but also attributable to platelet-mediated clot retraction) 30 minutes after MA. The LY30 parameter is calculated as loss of potential area under the TEG curve compared to a hypothetical tracing exhibiting no lysis in the same time period. The LY30 value is typically 0% to about 8%. The larger the LY30 value, the faster clot lysis (also called fibrinolysis) occurs.

When no fibrinolysis occurs, the amplitude value at the MA tracing stays constant or may decrease slightly due to clot retraction. However, as fibrinolysis occurs (e.g., in a healthy individual), the curve of the TEG tracing starts to decay. The resultant loss in potential area-under-the-curve in the 30 minutes following Maximum Amplitude in the TEG assay is the LY30 (see FIG. 1). LY30, the percentage of lysis 30 minutes after the maximum amplitude point (expressed as a percentage of the clot lysed) indicates the rate of coagulation characteristic.

It should be noted that modifications of the TEG assay can be performed.

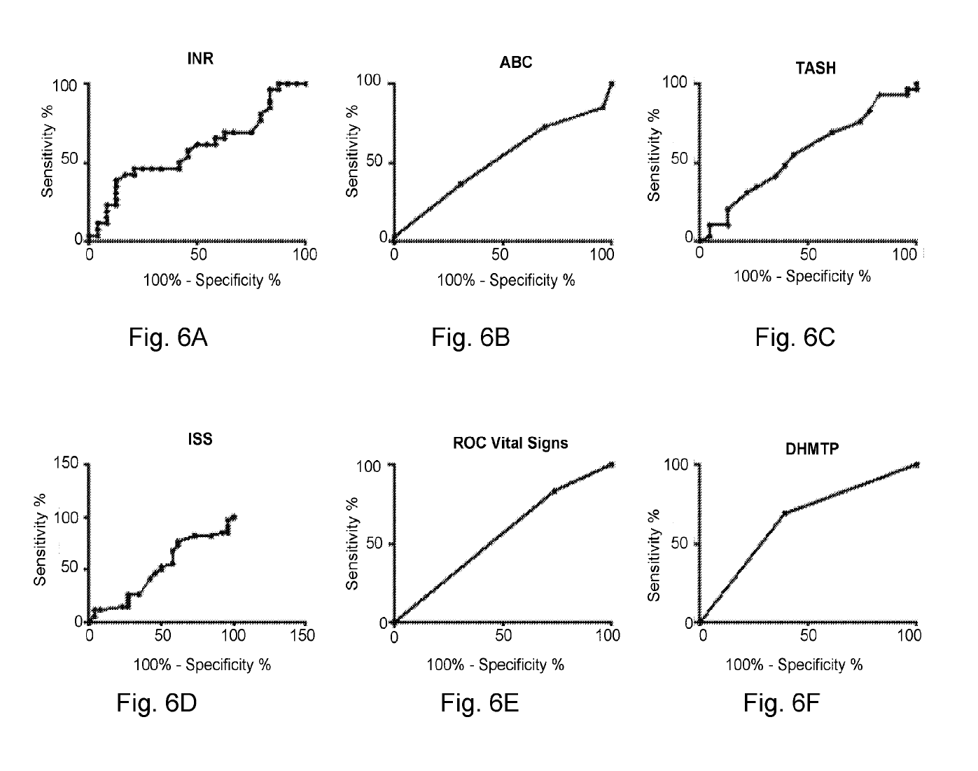

Another viscoelastic assay that can be used in accordance with various embodiments of the present invention is the thromboelastometry (“TEM”) assay. This TEM assay may be performed using the ROTEM Thromboelastometry Coagulation Analyzer (TEM International GmbH, Munich, Germany), the use of which is well known (See, e.g., Sorensen, B., et al., J. Thromb. Haemost., 2003. 1(3): p. 551-8. Ingerslev, J., et al., Haemophilia, 2003. 9(4): p. 348-52. Fenger-Eriksen C., et al. Br J Anaesth, 2005. 94(3): p. 324-9]. In the ROTEM analyzer, the blood sample is placed into a container (also called a cuvette or cup) and a cylindrical pin is immersed. Between pin and the interior wall of the container there is a gap of 1 mm which is bridged by the blood. The pin is rotated by a spring to the right and the left. As long as the blood is liquid (i.e., unclotted), the movement is unrestricted. However, when the blood starts clotting, the clot increasingly restricts the rotation of the pin with rising clot firmness. The pin is connected to an optical detector. This kinetic is detected mechanically and calculated by an integrated computer to the typical tracing curves (TEMogram) and numerical parameters (see FIGS. 6A and 6B).

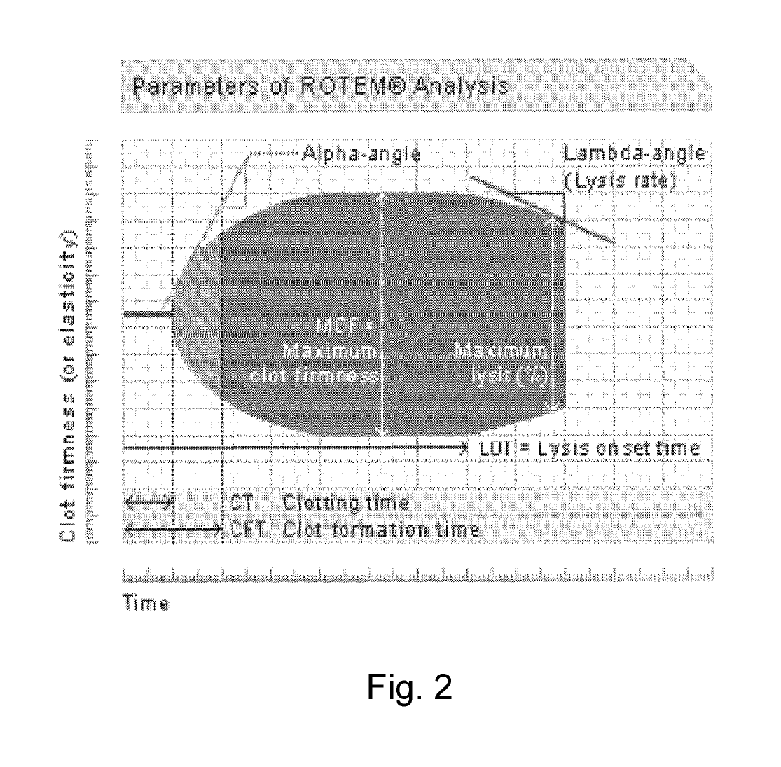

In the ROTEM Thromboelastometry Coagulation Analyzer, the movement of the pin can be monitored by a computer including a processor and a control program. The computer is operable on the electrical signal to create a hemostasis profile corresponding to the measured clotting process. Additionally, the computer may include a visual display or be coupled to a printer to provide a visual representation of the hemostasis profile (called a TEMogram. Such a configuration of the computer is well within the skills of one having ordinary skill in the art. As shown in FIG. 2, the resulting hemostasis profile (i.e., a TEM tracing curve) is a measure of the time it takes for the first fibrin strand to be formed, the kinetics of clot formation, the strength of the clot (measured in millimeters (mm) and converted to shear elasticity units of dyn/cm 2) and dissolution of clot. The descriptions for several of these measured parameters are as follows:

CT (clotting time) is the period of time of latency from the time that the blood was placed in the ROTEM analyzer until the clot begins to form. This CT time may be used as a non-limiting coagulation characteristic parameter reflective of clotting time in accordance with the methods described herein.

CFT (Clot formation time): the time from CT until a clot firmness of 20 mm point has been reached. This CFT time may be used as a non-limiting coagulation characteristic parameter reflective of clot formation in accordance with the methods described herein.

alpha-angle: The alpha angle is the angle of tangent at 2 mm amplitude. This alpha angle may be used as a non-limiting coagulation characteristic parameter reflective of clot formation in accordance with the methods described herein.

MCF (Maximum clot firmness): MCF is the greatest vertical amplitude of the trace. MCF reflects the absolute strength of the fibrin and platelet clot. If the blood sample tested has a reduced platelet function, this MCF is a function of mainly the fibrin bonding strength. The MCF value may be used as a non-limiting coagulation characteristic parameter reflective of clot strength in accordance with the methods described herein.

A10 (or A5, A15 or A20 value). This value describes the clot firmness (or amplitude) obtained after 10 (or 5 or 15 or 20) minutes and provide a forecast on the expected MCF value at an early stage. Any of these A values (e.g., A10) may be used as a non-limiting coagulation characteristic parameter reflective of clot strength in accordance with the methods described herein.

LI 30 (Lysis Index after 30 minutes). The LI30 value is the percentage of remaining clot stability in relation to the MCF value at 30 min after CT. This LI30 value may be used as a non-limiting coagulation characteristic value in accordance with the methods described herein. When no fibrinolysis occurs, the amplitude value at the MCF on a TEM tracing stays constant or may decrease slightly due to clot retraction. However, as fibrinolysis occurs (e.g., in a hypocoagulable state), the curve of the TEM tracing starts to decay. LI30 corresponds to the LY30 value from a TEG tracing. Thus, LI30 may be used as a non-limiting coagulation characteristic parameter reflective of clot lysis in accordance with the methods described herein.

ML (Maximum Lysis). The ML parameter describes the percentage of lost clot stability (relative to MCF, in %) viewed at any selected time point or when the test has been stopped. This ML value may be used as a non-limiting coagulation characteristic parameter reflective of clot lysis in accordance with the methods described herein.

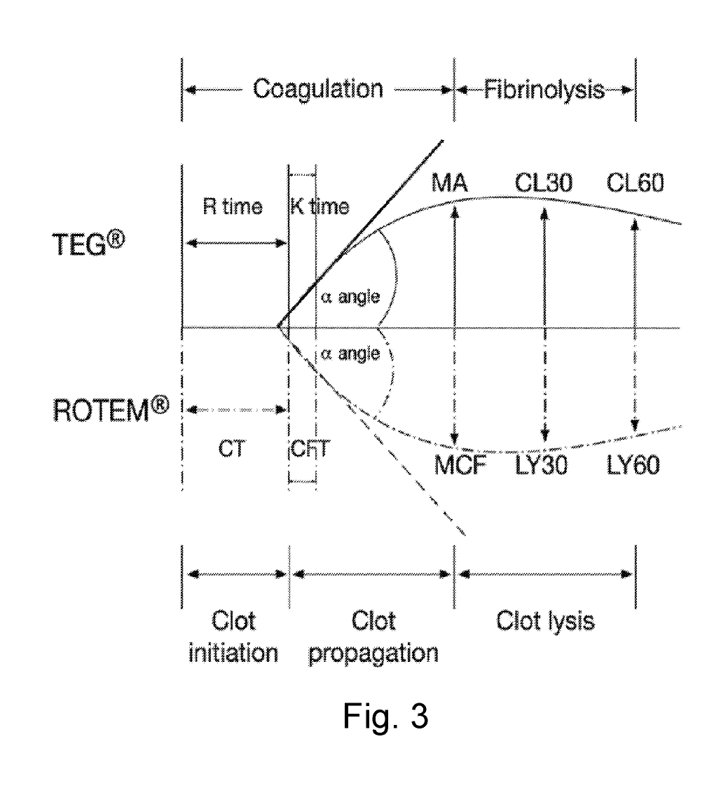

Thus, various parameters in TEG or TEM assays can be used as a coagulation characteristic parameter in accordance with the methods described herein. A TEG tracing and a TEM tracing are shown side by side in FIG. 3. For example, a third coagulation characteristic parameter reflective of clot strength includes the MA value in the TEG assay, and the MCF value in the TEM assay. The reaction time (R) in TEG (measured in seconds or minutes) and clotting time (CT) in TEM, which is the time until there is first evidence of clotting, are non-limiting examples coagulation characteristics reflective of clotting time. Clot kinetics (K, measured in minutes) is a coagulation characteristic parameter in the TEG assay indicating the achievement of clot firmness and thus is reflective of clot formation. The α (alpha) angle in both TEG and TEM is an angular measurement from a tangent line drawn to the curve of the TEG tracing or TEM tracing starting from the point of clot reaction time; thus, the α (alpha) angle is coagulation characteristic parameter that is reflective of clot formation and the kinetics of clot formation and development. (See Trapani, L. M. Thromboelastography: Current Applications, Future Directions”, Open Journal of Anesthesiology 3(1): Article ID: 27628, 5 pages (2013); and Kroll, M. H., “Thromboelastography: Theory and Practice in Measuring Hemostasis,” Clinical Laboratory News: Thromboelastography 36(12), December 2010; instruction manuals for the TEG instrument (available from Haemonetics, Corp.), and the instruction manual for the ROTEM instrument (available from TEM International GmbH), all of which documents are herein incorporated by reference in their entireties.

In some embodiments, the parameters (i.e., the coagulation characteristic parameters) are recorded by observation of different excitation levels of the sample as coagulation occurs. For example, where the container is a microfluidic cartridge, or a particular channel in the cartridge, the blood sample may be excited at a resonant frequency and its behavior observed by an electromagnetic or light source as coagulation occurs. In other embodiments the sample's coagulation characteristic parameter may be observed for changes with a light source without exciting the sample.

Because a single cartridge may have multiple containers (e.g., different channels in the cartridge), multiple patient samples can be analyzed at the same time (e.g., each patient sample is in a separate channel in the same microfluidic cartridge).

In a first aspect, the invention provides a method for identifying a patient as likely to have an onset of massive hemorrhage. The method comprises measuring a first coagulation characteristic parameter reflective of a clotting time in a sample of blood of the patient using a viscoelastic assay to obtain a first result, wherein, if the first result is positive, the patient is identified as likely to have an onset of massive hemorrhage. If the first result is negative, the method further comprises measuring a second coagulation characteristic parameter reflective of clot formation in a sample of blood of the patient using the viscoelastic assay to obtain a second result, wherein, if the second result is positive, the patient is identified as likely to have an onset of massive hemorrhage. If the second result is negative, the method further comprises measuring a third coagulation characteristic parameter reflective of clot strength in a sample of blood of the patient using the viscoelastic assay to obtain a third result, wherein, if the third result is positive, the patient is identified as likely to have an onset of massive hemorrhage. If the third result is negative, the method further comprises measuring a fourth coagulation characteristic parameter reflective clot lysis in a sample of blood of the patient using the viscoelastic assay to obtain a fourth result, wherein, if the fourth result is positive, the patient is identified as likely to have an onset of massive hemorrhage.

By “likely to have an onset of massive hemorrhage” is meant that a patient is more likely that not to have a massive hemorrhage necessitating a transfusion or resuscitation with blood (e.g., whole blood, platelets, plasma, etc.) The blood transfusion needed by a patient can be a massive transfusion protocol, but can also be a transfusion of a smaller amount of blood (e.g., one unit of packed red blood cells or one unit of fresh frozen plasma).

It should be noted that the sample of blood can be a sample of whole blood, any component of blood (e.g., platelet-reduced red blood cells or enriched platelets), or blood (or blood component) treated with an agent such as citrate, kaolin, tPA, etc.

Further, while the patient is typically a human patient, the methods described herein are applicable to any non-human animal including, without limitation, domesticated animals, such as cows, pigs, sheep, horses, chickens, cats, and dogs, and exotic animals (e.g., elephants, lions, ostriches, and whales).

FIG. 4 shows a flow chart diagrammatically depicting a non-limiting method of the invention. As shown in FIG. 4, if only one of the first, second, or third result is positive (e.g., the first result), the patient is identified as likely to have an onset of massive hemorrhage and there is no need to obtain any other result.

It should be noted that the order of the first result, second result, third result, and fourth result is not important. For example, the third result can be obtained first, and if it is positive, then the patient is identified as likely to have an onset of massive hemorrhage and there is no need to obtain any other result. Likewise, the second result can be obtained first, and if it is negative, and a fourth result is then obtained, with the fourth resulting being positive, the patient is identified as likely to have as an onset of massive hemorrhage.

Thus, in another aspect, the invention provides a method for identifying a patient as likely to have an onset of massive hemorrhage, comprising: measuring at least one of first coagulation characteristic parameter reflective of a clotting time in a sample of blood of the patient, a second coagulation characteristic parameter reflective of clot formation speed in a sample of blood of the patient using the viscoelastic assay to obtain a second result; a third coagulation characteristic parameter reflective of clot strength in a sample of blood of the patient using the viscoelastic assay to obtain a third result; and a fourth coagulation characteristic parameter reflective of clot lysis in a sample of blood of the patient using the viscoelastic assay to obtain a fourth result; wherein, a positive for at least one of the first result, second result, third result and fourth result identifies the patient as likely to have an onset of massive hemorrhage.

In various embodiments, the first coagulation characteristic parameter reflective of clotting time is an activated clotting time (ACT) value, a clotting time (CT) value, a reaction time (R) value, or a slight early split point (SP) value.

In various embodiments, the second coagulation characteristic reflective of clot formation is an α-angle value, a K value, or a CFT value.

In various embodiments, the third coagulation characteristic parameter reflective of clot strength is a maximum amplitude (MA) value, a maximum clot firmness (MCF) value, an A5 value, a A10 value, an A15 value, and an A20 value.

In various embodiments, the fourth coagulation characteristic parameter reflective of clot lysis is an LY30 value or an LI30 value.

In some embodiments of the methods described herein, the viscoelastic analysis is performed using a TEG thromboelastography analyzer system or in a ROTEM thromboelastometry analyzer system.

The invention is based, in part, on the examples described herein wherein it was found that derangements of TEG-derived coagulation characteristic parameters are powerful predictors of MH in patients suffering traumatic injury. The findings presented herein were valid both in a retrospective analysis of very high risk patients (those who triggered an MTP based upon clinician judgment) as well as a prospective analysis of a broader group of trauma activation patients whose blood sample for TEG was drawn at the earliest possible time to injury, usually in the field. The validity of this finding in the latter group is of great practical importance, as it suggests that very early TEG samples are useful for prediction of massive hemorrhage (and thus for guiding activation of a massive transfusion protocol and mobilization of other resources) even in a general trauma population where the pretest probability of MH is <10%. Coupled with recent advances in VHA technology which may allow placement of sophisticated coagulation analyzers in helicopters or ground ambulance units, these findings hold the promise of high-accuracy prediction of bleeding risk and transfusion requirements before the patient even arrives at the hospital.

Trauma scoring systems such as TASH, ABC, the ROCVS criteria and the massive transfusion protocol (MTP) trigger criteria of the Denver Health Medical Center (Denver, Colo.) were all designed to achieve this same goal using metrics available at the point of care. All of these scoring systems fell well short of the predictive value of any of the TEG-based parameters, generally having ROC plots not distinguishable from chance. Calculating these scores also requires multiple metrics (including a FAST exam), which require an experienced clinician to interpret. INR and ISS likewise had no significant predictive utility for MH.

It should be noted that TEG has some inherent limitations as well. TEG is, by its nature, more specific than sensitive for bleeding prediction, as TEG is insensitive to massive hemorrhage of purely mechanical (i.e. non-coagulopathic) cause. In a briskly bleeding patient who has not yet developed a coagulopathy the TEG will be normal, much as the hematocrit of a patient who is rapidly bleeding whole blood will remain normal until hemorrhagic shock is quite advanced. Moreover, individual TEG parameters each interrogate only certain aspects of the hemostatic system. Trauma induced coagulopathy (TIC) is a polymorphous entity, and while patients with the most severe TIC will eventually display a pan-coagulopathy affecting every stage of clot formation from enzymatic initiation to fibrinolysis, most patients display a heterogeneous mixture of more subtle and isolated disturbances of coagulation. It is precisely these patients, who are in early TIC, who must be accurately and rapidly diagnosed so that they can be treated with appropriate hemostatic resuscitation therapy.

Thus, in some embodiments, to accurately interpret the TEG, each of the four major parameters which describe the tracing shape can be analyzed sequentially (as in the GT95 parameter). FIG. 4 provides a flow chart showing four different parameters being analyzed sequentially. Note that although the first parameter is typically a parameter reflective of clotting time, the parameter reflective of clotting time does not need to be performed first. Rather, four parameters are performed sequentially, and if any of them results in a positive outcome, the analysis can be stopped because the patient is identified as likely to have an onset of massive hemorrhage. Consequently, the patient so identified can be administered blood or a blood product (e.g., packed red blood cells, whole blood, plasma, platelets, etc.).

In some embodiments, to accurately interpret the TEG, each of the four major parameters are analyzed in aggregate parameters. Some examples of these aggregate parameters are the four parameter TEG normalized sum (FPNS) parameter and the Angle+MA parameter described below.

Moreover, the results set forth below strongly suggest that a single TEG assay may not always be ideal, and the best predictive power comes from a combined use of the CR-TEG and CFF-TEG assays, with the CR-TEG generally being more useful for early TEG parameters (α-angle and MA and to a lesser extent ACT) while CFF-TEG is more useful for the analysis of the late TEG tracing features from MA to LY30. This is not surprising, as the platelet-inhibited basis of the CFF-TEG assay is more suited to detecting subtle derangements of fibrin structure and quality (e.g. factor XIII integration) as well as hyperfibrinolysis; however, the removal of platelet function masks the important contribution of platelets to clot growth which would normally be observed in the early TEG curve, as represented by R-time and α-angle. Neither of these assays extracts very good predictive power from the ACT/R-time parameters which quantitate the rate of the enzymatic initiation phase of clotting. While extreme prolongations of ACT or R are highly specific for coagulopathy, sensitivity of these parameters is extremely poor. This may be simply a function of their occurrence extremely early in the clotting process, before other aspects of the hemostatic system (e.g. fibrinogen concentration) may manifest themselves in contributing to the TEG signal. Alternatively, it may be that the very strong activators in the CR-TEG and CFF-TEG reagents may simply “wash out” a weak coagulopathy signal in this portion of the TEG tracing, much like the loss of detail in an overexposed photograph. This warrants further investigation using other TEG assays employing gentler clot activators.

A significant challenge in designing and interpreting the present study lies in properly defining the “disease positive” population, in the sense of building a two-by-two contingency table. In Examples I and II below, a transfusion requirement of 10 units of PRBCs in 6 hours (or death from hemorrhage prior to then) was used as the definition of MH. This somewhat arbitrary threshold left out several patients in Example II who might reasonably have been judged to have MH by other standards, such as one patient who received 8 units of PRBCs, 6 of fresh frozen plasma and a resuscitative thoracotomy. The very conservative, non-subjective and simple criteria was used in Example I and II to avoid over-fitting the predictive model to the limited clinical data in Examples I and II. Of course, the MH definition may be expanded. Note that the simple definition for MH, along with blinding of clinicians to the TEG data in the analysis in Example II, eliminated many forms of bias and enhanced the validity and applicability of the prognostic criteria developed herein.

Another ambiguity in Example II is whether the population of predictive interest is only patients with trauma induced coagulopathy (TIC) or all patients with massive hemorrhage (MH), whether coagulopathic or not. From a practical standpoint, this question seems to be resolving itself. While little progress has been made on rigorously defining clinical coagulopathy much less in deriving a gold standard biochemical assay for it, increasing bodies of evidence indicate that the relationship between MH and TIC is causally cyclic, and that the one may be essentially used as proxy for the other, owing to their entanglement.

Moreover, measurable signs of coagulopathy appear to be present in nearly every patient who is severely injured enough to eventual suffer MH, so early detection of subtle coagulopathy may indeed be the best means of evaluating all patients for their risk of major bleeding. This is best illustrated by the hypersensitivity to exogenous tPA seen in the LY30 parameter of the tissue plasminogen activator (tPA)-challenged citrated native TEG (TCN-TEG) in every patient who went on to have MH (100% sensitivity), even in the very early samples collected at the scene of injury. Interestingly, the TCN-TEG flagged as positive two patients with apparently normal initial conventional TEGs who later decompensated, displaying profound coagulopathy with associated MH. These findings suggest that a latent form of TIC exists in some patients, which can be revealed by challenge with exogenous tPA.

In sum, the results from Examples I and II below show that coagulation characteristic parameters derived from viscoelastic assays (e.g., TEG and TEM) are the best available predictors of MH in the severely injured trauma population. Thus, viscoelastic analysis of blood samples obtained in the field or upon arrival is probably the best basis for triggering a massive transfusion protocol (MTP) or other protocol for administering blood (e.g., transfusion of platelets, whole blood, etc) by identifying the patient as likely to have an onset of massive hemorrhage. The best combination of commercially available TEG assays for this task are the Citrated rapid TEG (CR-TEG), and the citrated functional fibrinogen TEG (CFF-TEG) assays both commercially available from Haemonetics, Corp. (Braintree, Mass.). The simplest application of the parameters extracted from these assays is the GT95 parameter, according to the manufacturer's instructions.

As discussed above, in some embodiments the methods described herein can be implemented clinically as a simple algorithm whereby the following parameters are analyzed sequentially. In some embodiments, the parameters are analyzed as they are reported by the evolving TEG tracings: Citrated rapid-TEG (CR-TEG) ACT (reflective of clotting time using CR TEG), α-angle (reflective of using citrated rapid TEG), and MA as well as citrated functional fibrinogen TEG (CFF-TEG) LY30. In some embodiments, thresholds of >152 seconds, <61.2 degrees, <49 mm and >2.5% should be applied to these parameters, respectively, and the test is scored positive if any of these four parameters is positive. If CFF is not available, the CR-TEG LY30 may be substituted, using an LY30 threshold of >3.9%. Note that the “Absolute MA” mode in the TEG 5000 software package may be selected in order to most accurately report LY30.

These recommendations are intended for application to very early blood samples either drawn in the field or at the time of admission, and are fundamentally prognostic in their utility. A positive GT95 test may be useful for triggering a massive transfusion protocol (MPT), or raising the clinician's index of suspicion for occult bleeding or coagulopathy and thus identifying a patient as likely to have an onset of massive hemorrhage. More work remains to be done to enhance these guidelines to direct the ongoing process of hemostatic resuscitation and the transfusion of specific blood product (e.g., fresh frozen plasma, platelets, whole blood, etc.).

Accordingly, in some embodiments, the result of a coagulation characteristic reflective of a clotting time is a positive result if the coagulation characteristic reflective of a clotting time result is greater than or equal to (or greater than or equivalent to) a 152 second when the coagulation characteristic reflective of a clotting time is an activated clotting time (ACT) value as measured by a thromoboelastography viscoelastic analysis assay.

As used herein, by “greater than or equal to” or “greater than or equivalent to” is meant that a value obtained from the indicated coagulation characteristic parameter is greater than or equal to the indicated value of the reference value (e.g., an ACT value result as measured by a thromoboelastography viscoelastic analysis assay) if the value obtained from the sample (e.g., a blood sample from the patient), had the sample been obtained using the indicated coagulation characteristic parameter (e.g., an ACT value result as measured by a thromoboelastography viscoelastic analysis assay), was greater than or equal to the reference value.

For example, a patient's blood may be assayed using thromboelastometry analysis, and a CT (clotting time) value obtained. If that same blood sample were to be assayed were assayed using thromoboelastography analysis and an ACT value obtained that is greater than or equal to 152 seconds (e.g., a value of 155 seconds is obtained), then the coagulation characteristic reflective of a clotting time is positive, and the patient is identified as being likely to have an onset of massive hemorrhage.

Of course, such a conversion from a CT value from thromboelastometry analysis to ACT value from thromoelastography analysis can be determined by charts or tables developed beforehand. This chart could be very easily constructed by any ordinarily skilled biologist or medical practitioner (e.g., a nurse, a physician, or a biological scientist), and would simply have a listing of all the values from the various metrics used as a coagulation characteristic reflective of a clotting time (e.g., an activated clotting time (ACT) value, a clotting time (CT) value, a reaction time (R) value, or an early split point (SP) value), obtained on the particular viscoelastic assay used (e.g., thromboelastometry at standard setting thromboelasography at standard setting) and indicate what value obtained is greater than or equal to the reference value of 152 seconds for an ACT value as measured by a thromoelastography analysis. The fidelity of the table can be improved, of course, upon increasing the number of patients whose samples are tested, and by standardizing other variables (e.g., age of the patient, gender, body mass index of the patient, blood pressure of the patient, heart rate of the patient, etc.).

In some embodiments, the result of a coagulation characteristic reflective of clot formation is a positive result if the coagulation characteristic reflective of clot formation result is less than or equal to (or less than or equivalent to) a 61.2 degrees when the coagulation characteristic reflective of clot formation is an α angle value as measured by a thromoboelastography viscoelastic analysis assay.

As used herein, by “less than or equal to” or “less than or equivalent to” is meant that a value obtained from the indicated coagulation characteristic parameter is less than or equal to the indicated value of the reference value (e.g., an α angle value result as measured by a thromoboelastography viscoelastic analysis assay) if the value obtained from the sample (e.g., a blood sample from the patient), had the sample been obtained using the indicated coagulation characteristic parameter (e.g., an α angle value result as measured by a thromoboelastography viscoelastic analysis assay), was less than or equal to the reference value.

For example, a patient's blood may be assayed using thromboelastometry analysis, and a CFT (clot formation time) value obtained. If that same blood sample were to be assayed were assayed using thromoboelastography analysis and an α angle value obtained that is less than or equal to 61.2 degrees is obtained (e.g., a value of 60.0 degrees is obtained), then the coagulation characteristic reflective of clot formation is positive, and the patient is identified as being likely to have an onset of massive hemorrhage.

Of course, such a conversion from a CFT value from thromboelastometry analysis to an α angle value from thromoelastography analysis can be determined by charts or tables developed beforehand. This chart could be very easily constructed by any ordinarily skilled biologist or medical practition, and would simply have a listing of all the values from the various metrics used as a coagulation characteristic reflective of clot formation (e.g., an α-angle value, a K value, or a CFT value), obtained on the particular viscoelastic assay used (e.g., thromboelastometry at standard setting or thromboelasography at standard setting) and indicate what value obtained is less than or equal to the reference value of 61.2 degrees for an α angle value as measured by a thromoelastography analysis. The fidelity of the table can be improved, of course, upon increasing the number of patients whose samples are tested, and by standardizing other variables (e.g., age of the patient, gender, body mass index of the patient, blood pressure of the patient, heart rate of the patient, etc.).

In some embodiments, the result of a coagulation characteristic reflective of clot strength is a positive result if the coagulation characteristic reflective of clot strength result is less than or equal to (or less than or equivalent to) 49 mm when the coagulation characteristic parameter reflective of clot strength is a maximum amplitude (MA) value as measured by a thromoboelastography viscoelastic analysis assay. In some embodiments, the coagulation characteristic reflective of clot strength is a maximum amplitude (MA) value, a maximum clot firmness (MCF) value, an A5 value, a A10 value, an A15 value, or an A20 value.

In some embodiments, the result of a coagulation characteristic reflective of clot lysis is a positive result if the coagulation characteristic reflective of clot lysis result is greater than or equal to (or greater than or equivalent to) 2.5% when the coagulation characteristic parameter reflective of clot lysis is an LY30 value as measured by a thromoboelastography viscoelastic analysis assay. In some embodiments, the coagulation characteristic parameter reflective of clot lysis is an LY30 value or an LI30 value.

In some embodiments, to accurately interpret the TEG results, each of the major parameters are analyzed in aggregate parameters.

In some embodiments, when two or more parameters are analyzed together, it is useful to normalize each result. One non-limiting method for normalizing the result is to divide the result by the mean of that coagulation characteristic parameter obtained in samples of blood of multiple trauma patients.

For example, the result of a coagulation characteristic reflective of clot formation may be determined to be equivalent to 64.4 degrees when the coagulation characteristic parameter reflective of clot formation is an α-angle value as measured by a thromoboelastography viscoelastic analysis assay. As described above, if this were a parameter being analyzed sequentially in accordance with one non-limiting embodiment of the invention, this 64.4 degrees result would be a negative result because it is greater than 61.2 degrees. However, if this 64.4 degrees result of a coagulation characteristic reflective of clot formation is analyzed together with the result of another coagulation characteristic parameter (e.g., a parameter reflective of clot lysis), the patient may nonetheless be identified as being likely to have an onset of massive hemorrhage.

In this non-limiting example, the result of 64.4 degrees when the coagulation characteristic parameter reflective of clot formation is an α-angle value as measured by a thromoboelastography viscoelastic analysis assay can be normalized by dividing the 64.4 degree value with the mean value of coagulation characteristic parameters reflective of clot formation from multiple trauma patients. The normalization can be fine-tuned, for example, by normalizing with a mean value from trauma patients all of the same age, and/or of the same gender, and/or having the same injury, and so forth.

The mean value of each coagulation characteristic parameter will depend on a variety of factors in addition to the status of the patient (e.g., age, gender, type of injury, etc.). Some of these factors include the type of viscoelastic analysis system used (e.g., thromboelastography or thromboelastometry), the type of instrument used (e.g., the TEG® 500 Thrombelastograph® thromboelastography analysis system sold by Haemonetics, Corp., Braintree, Mass. or the ROTEM® Delta thromboelastrometry analysis system sold by TEM International GmbH, Munich, Germany), and the parameters set on the instruments, e.g., using standard settings or using global TEG parameter derived at 90% specificity thresholds (GT90) as described below. Determining the mean value will, of course, be routine for any ordinarily skilled biologist or medical practitioner (e.g., a nurse, physician, or biological scientist such as a pharmacologist or technician).

In some embodiments, all four major parameters are analyzed together. Thus, in another aspect, the invention provides a method for identifying a patient as likely to have an onset of massive hemorrhage, comprising: (a) obtaining a normalized first result by: (i) measuring a first coagulation characteristic parameter reflective of a clotting time in a sample of blood of the patient using a viscoelastic assay to obtain a first result, and (ii) dividing the first result by a mean of the first coagulation characteristic parameter in samples of blood of trauma patients using the viscoelastic assay to obtain the normalized first result; (b) obtaining a normalized second result by: (i) measuring a second coagulation characteristic parameter reflective of clot formation in a sample of blood of the patient using the viscoelastic assay to obtain a second result; and (ii) dividing the second result by a mean of the second coagulation characteristic parameter in samples of blood of trauma patients using the viscoelastic assay to obtain the normalized second result; (c) obtaining a normalized third result by: (i) measuring a third coagulation characteristic parameter reflective of clot strength in a sample of blood of the patient using the viscoelastic assay to obtain a third result; and (ii) dividing the third result by a mean of the third coagulation characteristic parameter in samples of blood of trauma patients using the viscoelastic assay to obtain the normalized third result; (d) obtaining a normalized fourth result by: (i) measuring a fourth coagulation characteristic parameter reflective of clot formation speed time in a sample of blood of the patient using the viscoelastic assay to obtain a fourth result; and (ii) dividing the second result by a mean of the fourth coagulation characteristic parameter in samples of blood of trauma patients using the viscoelastic assay to obtain the normalized fourth result; and (e) obtaining a value by subtracting the sum of the normalized second result and the normalized third result from the sum of the normalized first result and the normalized fourth result; wherein a value that is greater than the threshold value established between healthy volunteers and positive control test subjects at a particular site of usage identifies the patient as likely to have an onset of massive hemorrhage.

In some embodiments, the value in a patient that is greater by about 1% than the threshold value established between healthy volunteers and positive control test subjects at a particular site of usage identifies the patient as likely to have an onset of massive hemorrhage. In some embodiments, the value in a patient that is greater by about 3% than the threshold value established between healthy volunteers and positive control test subjects at a particular site of usage identifies the patient as likely to have an onset of massive hemorrhage. In some embodiments, the value in a patient that is greater by about 5% than the threshold value established between healthy volunteers and positive control test subjects at a particular site of usage identifies the patient as likely to have an onset of massive hemorrhage. In some embodiments, the value in a patient that is greater by about 10% than the threshold value established between healthy volunteers and positive control test subjects at a particular site of usage identifies the patient as likely to have an onset of massive hemorrhage. In some embodiments, the value in a patient that is greater by about 20% than the threshold value established between healthy volunteers and positive control test subjects at a particular site of usage identifies the patient as likely to have an onset of massive hemorrhage. In some embodiments, the value in a patient that is greater by about 30% than the threshold value established between healthy volunteers and positive control test subjects at a particular site of usage identifies the patient as likely to have an onset of massive hemorrhage. In some embodiments, the value in a patient that is greater by about 50% than the threshold value established between healthy volunteers and positive control test subjects at a particular site of usage identifies the patient as likely to have an onset of massive hemorrhage. In some embodiments, the value in a patient that is greater by about 100% than the threshold value established between healthy volunteers and positive control test subjects at a particular site of usage identifies the patient as likely to have an onset of massive hemorrhage.

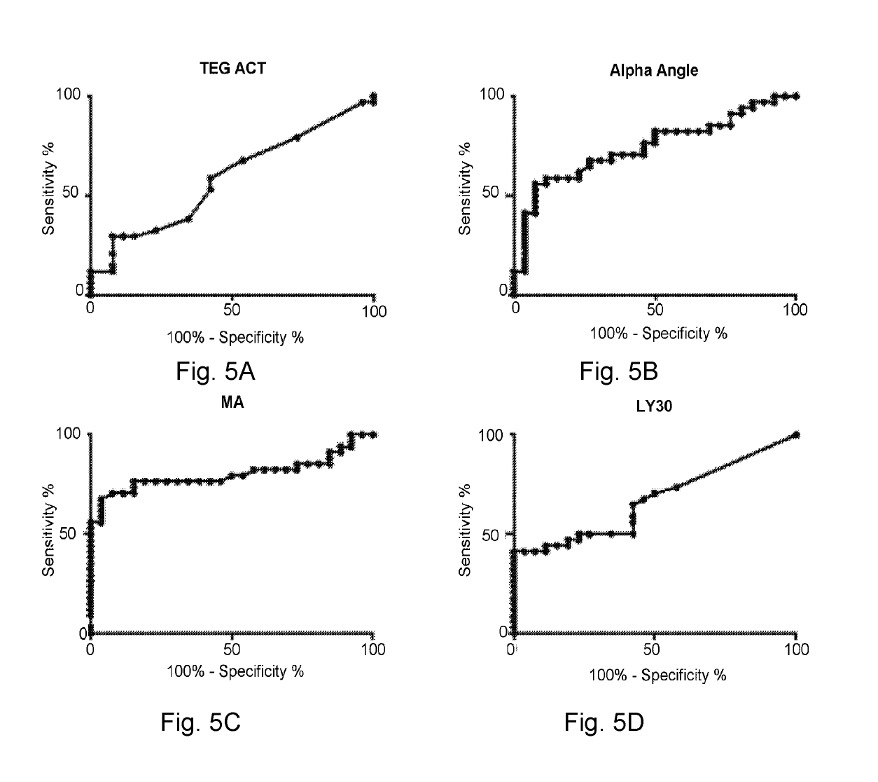

As used herein, by “threshold value” is simply the value that lies between the values of healthy volunteers and the values of positive control subjects, as determined by the routinely skilled clinician. Note that a positive control subject is simply an individual that is clearly bleeding to death. Generally, a receiver operating characteristic (ROC) curve is used to determine threshold value taking into account the sensitivity and specificity of the particular assay used. See, e.g., the ROC curves for TEG-ACT (FIG. 5A), Alpha Angle (FIG. 5B), MA (FIG. 5C), and LY30 (FIG. 5D).

By a particular “site of usage” is simply meant the site that the viscoelastic assay is performed, because the values obtained at the site will take into account the demographics of the population (e.g., age, race, gender, body mass index, etc.) and environmental considerations (e.g., altitude, air quality). For example, a site of usage may be a hospital in Denver, Colo., USA. A “healthy volunteer” (or “healthy individual”) is defined a healthy organism (e.g., a healthy human) who is uninjured and is otherwise healthy (e.g., free of disease such as cancer and of a normal body mass index). For example, if the patient is a human, a healthy human individual is between the ages of 14 to 44, and may be male or female.

In one non-limiting example of various embodiments of the invention, a thirty year old male gun shot victim may be brought into the emergency room. While he is bleeding, it may be unclear whether he is likely to have an onset of massive hemorrhage. A sample of whole blood may be taken when the patient is admitted and subjected to TEG viscoelastic analysis. Four parameters, namely an activated clotting time (ACT) value (which is reflective of clotting time), an α-angle value (which is reflective of clot formation), a maximum amplitude (MA) value (which is reflective of clot strength) and an LY30 value (which is reflective of clot lysis) are taken. Each of these four parameters is normalized by dividing each value with the mean value of that parameter in a population of multiple trauma patients. For example, the ACT value is divided by the mean of ACT values from multiple trauma patients. The normalized α-angle value and the normalized MA value are added together to obtain a first sum. The normalized ACT value and the normalized LY30 value are added together to obtain a second sum. The second sum is then subtracted from the first sum to obtain a number. If that number is greater than the threshold value established between healthy volunteers and positive control test subjects at a particular site of usage the gun shot victim is identified as likely to have an onset of massive hemorrhage, and will be administered blood (e.g., platelets or whole blood). In some embodiments, the administration route is intravenous. Conversely, if the number is lower than the threshold value established between healthy volunteers and positive control test subjects at a particular site of usage the patient is identified as not likely to have an onset of massive hemorrhage and thus will not be administered blood.

In some embodiments, the number in a patient that is greater by about 1% than the threshold value established between healthy volunteers and positive control test subjects at a particular site of usage identifies the patient as likely to have an onset of massive hemorrhage. In some embodiments, the number in a patient that is greater by about 3% than the threshold value established between healthy volunteers and positive control test subjects at a particular site of usage identifies the patient as likely to have an onset of massive hemorrhage. In some embodiments, the number in a patient that is greater by about 5% than the threshold value established between healthy volunteers and positive control test subjects at a particular site of usage identifies the patient as likely to have an onset of massive hemorrhage. In some embodiments, the number in a patient that is greater by about 10% than the threshold value established between healthy volunteers and positive control test subjects at a particular site of usage identifies the patient as likely to have an onset of massive hemorrhage. In some embodiments, the number in a patient that is greater by about 20% than the threshold value established between healthy volunteers and positive control test subjects at a particular site of usage identifies the patient as likely to have an onset of massive hemorrhage. In some embodiments, the number in a patient that is greater by about 30% than the threshold value established between healthy volunteers and positive control test subjects at a particular site of usage identifies the patient as likely to have an onset of massive hemorrhage. In some embodiments, the number in a patient that is greater by about 50% than the threshold value established between healthy volunteers and positive control test subjects at a particular site of usage identifies the patient as likely to have an onset of massive hemorrhage. In some embodiments, the number in a patient that is greater by about 100% than the threshold value established between healthy volunteers and positive control test subjects at a particular site of usage identifies the patient as likely to have an onset of massive hemorrhage. In some embodiments, if the number in the patient is greater than 10, or greater than 25, or greater than 50, or greater than 75, or greater than 100, than the threshold value established between healthy volunteers and positive control test subjects at a particular site of usage, then the gun shot victim is identified as likely to have an onset of massive hemorrhage, and will be administered blood (e.g., platelets or whole blood)

In some embodiments, the number in a patient that is lower by about 1% than the threshold value established between healthy volunteers and positive control test subjects at a particular site of usage identifies the patient as not likely to have an onset of massive hemorrhage. In some embodiments, the number in a patient that is lower by about 3% than the threshold value established between healthy volunteers and positive control test subjects at a particular site of usage identifies the patient as not likely to have an onset of massive hemorrhage. In some embodiments, the number in a patient that is lower by about 5% than the threshold value established between healthy volunteers and positive control test subjects at a particular site of usage identifies the patient as not likely to have an onset of massive hemorrhage. In some embodiments, the number in a patient that is lower by about 10% than the threshold value established between healthy volunteers and positive control test subjects at a particular site of usage identifies the patient as not likely to have an onset of massive hemorrhage. In some embodiments, the number in a patient that is lower by about 20% than the threshold value established between healthy volunteers and positive control test subjects at a particular site of usage identifies the patient as not likely to have an onset of massive hemorrhage. In some embodiments, the number in a patient that is lower by about 30% than the threshold value established between healthy volunteers and positive control test subjects at a particular site of usage identifies the patient as not likely to have an onset of massive hemorrhage. In some embodiments, the number in a patient that is lower by about 50% than the threshold value established between healthy volunteers and positive control test subjects at a particular site of usage identifies the patient as not likely to have an onset of massive hemorrhage. In some embodiments, the number in a patient that is lower by about 100% than the threshold value established between healthy volunteers and positive control test subjects at a particular site of usage identifies the patient as not likely to have an onset of massive hemorrhage.

This identification allows the patient to avoid the attendant risks associated with being administered blood (e.g., infection, blood borne illnesses, immune response to non-self MEW on the donor white blood cells, etc.).

In some embodiments, two coagulation characteristic parameters are used in aggregate to determine if a patient is like to have an onset of massive hemorrhage. For example, when the coagulation characteristic parameter reflective of clot formation and the coagulation characteristic parameter reflective of clot strength of a patient being examined are larger than the mean values of those parameters from multiple trauma patients, examined patient is identified as being likely to have an onset of massive hemorrhage. Since, as was discussed above, a positive result from any parameter identifies the patient as likely to have an onset of massive hemorrhage and thus require a blood transfusion, these two parameters themselves can be employed to identify such a patient. Thus, in another aspect, the invention provides a method for identifying a patient as likely to have an onset of massive hemorrhage, comprising: (a) obtaining a normalized first result by: (i) measuring a first coagulation characteristic parameter reflective of clot formation in a sample of blood of the patient using the viscoelastic assay to obtain a first result; and (ii) dividing the second result by a mean of the first coagulation characteristic parameter in samples of blood of trauma patients using the viscoelastic assay to obtain the normalized first result; (b) obtaining a normalized second result by: (i) measuring a second coagulation characteristic parameter reflective of clot strength in a sample of blood of the patient using the viscoelastic assay to obtain a second result; and (ii) dividing the second result by a mean of the second coagulation characteristic parameter in samples of blood of trauma patients using the viscoelastic assay to obtain the normalized second result; and (c) adding the normalized first result to the normalized second result to obtain a sum, where a sum that is greater than the threshold value established between healthy volunteers and positive control test subjects at a particular site of usage identifies the patient as likely to have an onset of massive hemorrhage.

In some embodiments, the sum from the patient that is greater by about 1% than the threshold value established between healthy volunteers and positive control test subjects at a particular site of usage identifies the patient as likely to have an onset of massive hemorrhage. In some embodiments, the sum from the patient that is greater by about 3% than the threshold value established between healthy volunteers and positive control test subjects at a particular site of usage identifies the patient as likely to have an onset of massive hemorrhage. In some embodiments, the sum from the patient that is greater by about 5% than the threshold value established between healthy volunteers and positive control test subjects at a particular site of usage identifies the patient as likely to have an onset of massive hemorrhage. In some embodiments, the sum from the patient that is greater by about 10% than the threshold value established between healthy volunteers and positive control test subjects at a particular site of usage identifies the patient as likely to have an onset of massive hemorrhage. In some embodiments, the sum from the patient that is greater by about 20% than the threshold value established between healthy volunteers and positive control test subjects at a particular site of usage identifies the patient as likely to have an onset of massive hemorrhage. In some embodiments, the sum from the patient that is greater by about 30% than the threshold value established between healthy volunteers and positive control test subjects at a particular site of usage identifies the patient as likely to have an onset of massive hemorrhage. In some embodiments, the sum from the patient that is greater by about 50% than the threshold value established between healthy volunteers and positive control test subjects at a particular site of usage identifies the patient as likely to have an onset of massive hemorrhage. In some embodiments, the sum from the patient that is greater by about 100% than the threshold value established between healthy volunteers and positive control test subjects at a particular site of usage identifies the patient as likely to have an onset of massive hemorrhage. In some embodiments, the sum that is greater than 10, or greater than 25, or greater than 50, or greater than 75, or greater than 100 than the threshold value established between healthy volunteers and positive control test subjects at a particular site of usage identifies the patient as likely to have an onset of massive hemorrhage.

In some embodiments, the where a sum that is greater than 10, or greater than 25, or greater than 50, or greater than 75, or greater than 100 identifies the patient as likely to have an onset of massive hemorrhage. In some embodiments, the first coagulation characteristic parameter reflective of clot formation is an α-angle value, a K value, or a CFT value. In some embodiments, the second coagulation characteristic parameter reflective of clot strength is a maximum amplitude (MA) value, a maximum clot firmness (MCF) value, an A5 value, a A10 value, an A15 value, or an A20 value.

The following examples are provided which are meant to illustrate but not limit the invention described herein.

### Examples I and II

Study Design

This observational study was conducted on two distinct patient groups, in order to develop and then independently test prognostic criteria for prediction of MH using the TEG platform. This methodology is conceptually analogous to the process of machine learning, wherein a heuristic system is first exposed to “training data” where outcomes are known and then the algorithms so developed are challenged for validity by application to a new set of “test data” where the outcomes are not known beforehand. This Example 1 describes the test data or retrospective patient group in this study. Example II below describes the training data or prospective patient group in this study.

### Example 1

The clinical lab Rapid Thrombelastography (R-TEG) data collected from all high-risk patients (age 18 or above and non-pregnant) between September 2010 and March 2014, for whom an MTP was triggered, was retrospectively analyzed to determine if any TEG parameters could be used to improve upon clinician judgment in terms of prediction of actual massive transfusion requirement (i.e. MH). These samples were drawn in the hospital prior to MTP activation. Patients whose first R-TEG was drawn more than 2 hours after injury were excluded from the study, as were transfers and patients receiving a transfusion prior to their first TEG. This “training” data set was used to develop trigger thresholds within existing R-TEG parameters for predicting massive hemorrhage and to develop novel synthetic parameters.

### Example II

The methods described above were then applied prospectively to a “test” data set comprised of all high-level trauma activation patients (non-pregnant adults) admitted to our center from May to December of 2014, to determine the generalizability of these trigger thresholds to a broader, but inherently lower-risk trauma population. Application of the original parameters to the second, test population also allowed for further refinement and expansion of the TEG-based parameters for prediction of massive hemorrhage. Blood samples for this group were drawn at the earliest possible time after injury, generally in the field by the responding ground ambulance crew, but occasionally upon ED arrival, if IV access was not obtainable in the field. Clinicians were blinded to the TEG results in the prospective patient group.

Outcomes for Examples I and II were analyzed in a binary manner with patients separated into two categories: those with major hemorrhage and those without. “Massive hemorrhage” (MH) is defined as the requirement for 10 or more units of PRBCs within the 6 hours after injury, or death from hemorrhage prior to that time point (i.e., 6 hours after injury).

Thrombelastography (TEG)

For Example I, in the retrospective patient group, R-TEG was run on unpreserved samples within 4 minutes of collection on the TEG 5000 analyzer platform, according the manufacturer's instructions (Haemonetics Corp., Braintree, Mass., USA). The first available sample obtained after hospital admission, but obtained no later than 2 hours after injury and prior to MTP activation, was used for the TEG assay.

For Example II, in the prospective patient group, samples were collected in the field by the ground ambulance crews (or occasionally in the ED if IV access was unavailable) and thus were collected in tubes containing a 3.2% citrate preservative. These samples were run within 30 minutes of collection in our research facility. Citrated R-TEG (CR-TEG), citrated functional fibrinogen TEG (CFF-TEG), and tissue plasminogen activator (tPA)-challenged citrated native TEG (TCN-TEG) were run on the TEG 5000 analyzer after reactivation of the citrated sample with a standard calcium chloride solution, according to the manufacturer's instructions.

For both Examples I and II, R-TEG (i.e., non-citrated) and CR-TEG (citrated) are native whole blood activated with a mixture of tissue factor and kaolin (stimulating both the extrinsic and intrinsic pathways). CFF-TEG is activated with a low dose of tissue factor in the presence of the gpIIb-IIIa inhibitor abciximab (ReoPro) which effectively blocks platelet binding to the fibrin clot and thus yields a value for clot strength dependent on the level of functional fibrinogen alone. TCN-TEG is run as native whole blood (i.e. no activator other than contact with the TEG cup and the atmosphere) but is run in the presence of 75 ng/mL of human single-chain tPA (available from Molecular Innovations, Novi, Mich., USA).

For Examples I and II, the TEG parameters initially evaluated included the split point (SP), reaction time (R), TEG activated clotting time (ACT), kinetic time (K), α-angle, maximum amplitude (MA), the calculated viscoelastic clot strength (G-value), and the clot lysis 30 minutes after MA (LY30), as shown in FIG. 1. SP is the time from initiation to when the TEG tracing diverges from the time (x) axis. R is the time until this divergence reaches the arbitrary value of 2 mm. ACT is a calculated value based entirely upon R, and is reported in lieu of R for R-TEG and CR-TEG as it is intended to be analogous to a traditional ACT used for heparin titration, the original design use of the R/CR-TEG assays. SP, R and ACT all provide information regarding the soluble enzymatic phase of clot initiation and these times are prolonged in coagulopathic states attributable to inhibited catalysis. K is time until the amplitude of the TEG tracing reaches the arbitrary value of 20 mm and α-angle is the angle from the time (x) axis to the tangent ray to the rising portion of the TEG tracing, drawn from the SP. Both K and α-angle provide information regarding the kinetics of clot growth and under most conditions are chiefly dependent on the available concentration of fibrinogen and, to a lesser extent, on platelet activity. MA reflects the final, maximal clot strength and G is simply a transformation of MA from the arbitrary units of mm (of tracing amplitude) into units of dynes/cm², which are intended to approximate the tensile strength of the clot. As SP, R and ACT bear a trivial relationship to each other, only ACT was analyzed for R/CR-TEG and R was analyzed for CFF, as these are the default values reported for these assays by the TEG 500 software. As K and α-angle are also trivially related to each other and as K is not reported at all in cases of severe coagulopathy when amplitude never reaches 20 mm, only α-angle was analyzed and K was discarded. Lastly, as G is a calculated value derived solely from MA, only MA was analyzed, and G was discarded, as it encoded no additional information.

Statistical Methods

Data for Examples I and II was analyzed using the Prism software package (GraphPad Prism version 6.0f for Mac OS X, GraphPad Software, La Jolla, Calif. USA). Receiver operating characteristic (ROC) curves were constructed for prediction of MH by each of the existing reported TEG parameters, and the area under the curve (AUC) reported. Parameters bearing a trivial or calculated relationship to other parameters were excluded as redundant (see “Thrombelastography”, above). TEG ACT, α-angle, MA and LY30 were retained for R-TEG and CR-TEG. For CFF-TEG, R was used in place of ACT. Optimized sensitivity and specificity pairs (i.e. nearest the upper left-hand corner of the ROC plot) were reported for each tested parameter.

Synthetic parameters were subsequently created to improve upon the performance of the isolated TEG parameters. This was done using two methodologies. First a “global TEG” parameter was constructed as a sequence of Boolean “or” statements for positive results of each tested parameter at a given threshold of specificity (i.e. if ACT or α-angle or MA or LY30 are positive, then test is positive) and is referred to as the “global TEG” parameter at a given specificity. A specificity cutoff of >90% was chosen for construction of this parameter in the training data. This was applied to the test data and subsequently refined for this group by using a 95% specificity cutoff, which was deemed more optimal owing to the lower pretest probability of MH in the test data patient cohort. The second methodology of parameter synthesis consisted of summing the four parameters ACT, α-angle, MA and LY30, with each parameter weighted by normalization to the mean of that parameter within the data set. MA and α-angle were given a positive sign in this summation function and ACT and LY30, negative, reflecting that fact that greater magnitudes of MA and α-angle are associated with more robust coagulation (i.e. TIC less likely), whereas larger values of ACT and LY30 reflect worse coagulopathy. Thus, overall, the four-parameter TEG normalized sum (FPNS) reports a higher likelihood of MH at smaller or negative values, again determined by plotting the ROC curve. During the optimization process, it was determined that in some cases use of fewer than four parameters (i.e. weighting some parameters with a zero scalar) achieved better predictive power and these normalized sums (e.g. α-angle plus MA or MA minus LY30) were analyzed by ROC curve in the same manner. Binary variables had ROC curves constructed as well for the purpose of convenient graphical representation, but naturally yielded only a single sensitivity/specificity pair with an AUC disproportionately low compared to the accuracy of the test compared to tests with more granular readout variables.

Other Metrics

During the period of enrollment of the retrospective patient group, other metrics with possible predictive value for MH were evaluated. These included the conventional coagulation test, prothrombin time (PT) expressed as an international normalized ratio (INR) and the routinely computed injury severity score (ISS). In addition to these single metrics, composite trauma scores were calculated. The Assessment of Blood Consumption (ABC) score is calculated by assigning the patient a score of 1 for each of the following four binary criteria: (1) penetrating mechanism, (2) positive focused assessment sonography for trauma (FAST), (3) admission systolic blood pressure (SBP) ≦90 mm Hg, and (4) arrival heart rate (HR) ≧120 (Nunez et al., Journal of trauma. 66(2):346-52, 2009). The Trauma-Associated Severe Hemorrhage (TASH) is calculated from scoring eight parameters (hemoglobin, base deficit, SBP, HR, FAST exam, pelvic and long bone fractures) yielding a score between 0 and 28. The details of this calculation are described in Yucel et al., Journal of trauma. 60(6):1228-36; discussion 36-7, 2006, incorporated herein by reference. The Resuscitation Outcomes Consortium vital signs (ROCVS) criteria were also tested as a predictor of MH, as they were initially designed to predict patients likely to benefit from enrolment in studies of hemostatic resuscitation agents using metrics available in the field. ROCVS criteria consist of either an SBP ≦70 mmHg or 71-90 mmHg with an accompanying heart rate ≧108 (Bulger et al., Annals of surgery. 2011 March; 253(3):431-41, 2011). Lastly we evaluated the newly revised objective trigger criteria for Denver Health Medical Center's MTP (DHMTP) which consists of meeting ROCVS criteria plus any of the following: penetrating torso injury, unstable pelvic fracture, or FAST positive in >1 scan region. ROCVS and DHMTP results were expressed as either a 1 for those patients meeting criteria or a 0 for those who do not, and the analysis conducted as for TEG parameters above.

Results

Patients

In the retrospective group in Example I, 111 patients were enrolled in the original study of MTP-activating patients, of which 51 (46%) actually went on to receive a massive transfusion. After excluding patients transferred from another institution and any patients having received blood products prior to their first R-TEG, 60 patients remained who received their first R-TEG within two hours of injury. Of these, 34 (57%) suffered MH. The median age in the retrospective group in Example I was 39 years (IQR 28-53); the group was 70% male, 32% with a penetrating mechanism and the median ISS was 30.

The prospective group of Example II consisted of 85 trauma activation patients, of whom 8 (9.4%) suffered massive hemorrhage. 84% were male, with a median age of 35 (IQR 27-48), 45% with a penetrating mechanism. There was an overall 12% mortality rate in the prospective trauma activation group, with 8 patients requiring resuscitative thoracotomy (of whom 4 survived).

### Example I: Prediction of Massive Hemorrhage in the Retrospective Group (Training Data Set)

Resuscitation Outcomes Consortium (ROC) plots were constructed for four basic R-TEG parameters: ACT (reflective of clotting time), α-angle (reflective of clot formation), MA (reflective of clot strength) and LY30 (reflective of clot lysis) and compared to those for the various trauma scoring systems as well as INR and ISS. For the four TEG parameters, AUC for the ROC plots were: 0.59 (p=0.25) for ACT; 0.74 (p=0.0013) for α-angle; 0.80 (<0.0001) for MA; and 0.67 (p=0.025) for LY30. ACT was optimized at a threshold of >171 seconds with a sensitivity of 29.4% and specificity or 92.3% but its ROC curve was not significantly different from the line of identity. α-angle was optimized at <63 degrees with 58.8% sensitivity and 84.6% specificity. MA was optimized at <49 mm with 76.5% sensitivity and 88.5% specificity. LY30 was optimized at >5.0% degrees with 41.2% sensitivity and 100% specificity (see FIGS. 5A-5D).

For the ROC plots of the trauma scoring systems AUCs were: 0.52 (p=0.81) for ABC; 0.56 (p=0.46) for TASH; 0.55 (p=0.56) for the Resuscitation Outcomes Consortium vital signs standard (ROCVSS); and 0.65 (p=0.067) for the new DHMTP trigger criteria. The AUC was 0.59 for INR (p=0.28) and 0.50 (p=0.95 for ISS). No optimized threshold for ABC, TASH or ISS was determinable as the ROC plots crossed the line of identity at random. INR was optimized for prediction at a threshold of >1.8 with 46.2% sensitivity and 79% specificity, but its ROC plot was likewise not statistically significantly different from the line of identity. For the binary variables, ROCVS was 83.3% sensitive and 29.1% specific and DHMTP criteria were 69% sensitive and 60.9% specific for MH prediction; however, plotted as a ROC curve, neither test was significantly different from the line of identity (see FIGS. 6A-6F).

Lastly, the synthetic TEG parameters were calculated. The global TEG parameter was designed based upon the observation that single TEG parameters' ROC curves were shaped such that high levels of specificity were obtainable, but sensitivity was not. This is self evident, as a particular patient may have one aspect of TIC, but not others, yet even one element of coagulopathy (e.g. hyperfibrinolysis) may be enough to provoke MH. Thus, the global TEG parameter is a binary variable, which reports positive (i.e. “1”) if any of the four basic TEG parameters is positive. The threshold for each individual parameter was set arbitrarily at the relatively stringent value of 90% as it was expected that this methodology would tend to lower specificity as it raised sensitivity. AUC for the global TEG parameter, at 90% specificity for each of its components (GT90), was 0.77 (p=0.0004), with a sensitivity of 76.5% and a specificity of 76.9% for MH prediction.

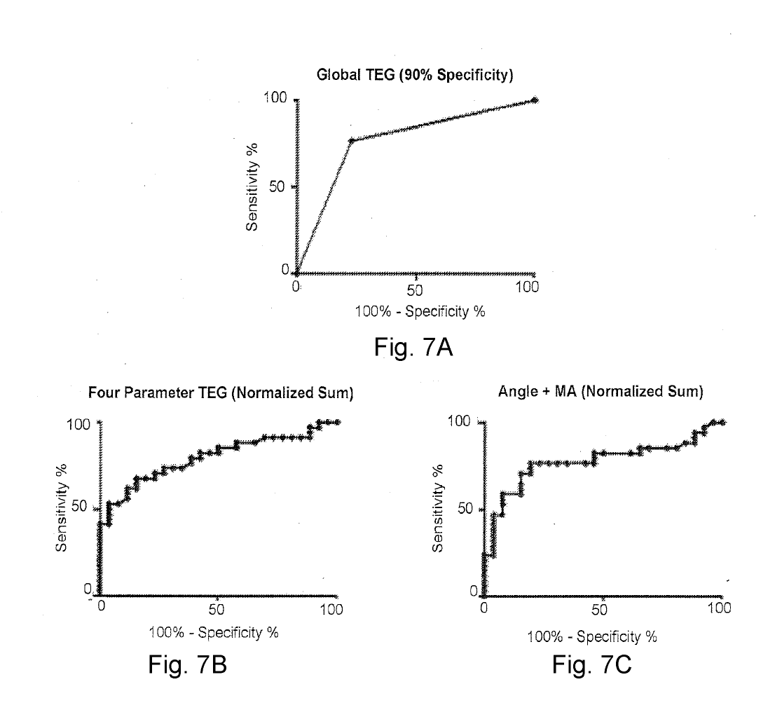

The continuous composite normalized parameter, FPNS, was calculated as described above, with scalars of −162.5, 59.5, 46.3, and −11.5 for ACT, α-angle, MA and LY30 respectively. The AUC for the FPNS was 0.80 (p<0.00001); and 0.77 (p=0.0003) for the simplified normalized sum of α-angle and MA (Angle+MA). FPNS had an optimized threshold of <1.28 with a sensitivity of 73.5% and a specificity of 73.1% and Angle+MA was optimized for prediction of MH at <2.22 with 76.5% sensitivity and 80.8% specificity (See FIGS. 7A-7C).

### Example II: Prediction of Massive Hemorrhage in the Prospective Group (Test Data Set)

The findings from the retrospective group were next applied, prospectively, to a new set of patients to test the validity of the TEG-based thresholds for MH prediction learned from “training” with the initial data set.

Several differences between the two data sets required modification of the tests developed with the training data set.

First, the prospective samples were collected with a citrate preservative and so might not be exactly comparable to the unpreserved retrospective samples, so ROC curves were constructed anew for all four TEG parameters, and scalars for the FPNS recalculated for the prospective patient group.

Secondly, an improvement to the TEG 5000 software algorithm in the intervening time period provided a more accurate calculation of both MA and LY30 with the general effect of slightly increasing both. This amplification effect, while improving sensitivity for fibrinolysis, also had the confounding effect of amplifying the platelet-mediated clot retraction phenomenon, which mimics fibrinolysis in the TEG tracing and creates false positives. This confounding effect was overcome by confirming fibrinolysis with a CFF-TEG run in parallel, wherein platelet participation in the clot architecture is blocked with a gpIIb-IIIa inhibitor.

Lastly, selection criteria for the prospective group included a much broader population (all trauma team activations) than the retrospective study (all MTP activations), with an inherently lower pretest probability of MH, therefore higher levels of specificity would be desirable to achieve clinical utility in this group, therefore a global TEG parameter, based upon 95% specificity for each of the comprising parameters, was re-derived (GT95).

In addition to CR-TEG, as two other TEG based assays were available for use in the prospective patient population, CFF-TEG and the tPA-challenged TCN-TEG, these technologies were also tested for predictive utility for massive hemorrhage (MH). The CFF-TEG LY30 parameter was explicitly substituted for the CR-TEG LY30 parameter in the synthesis of the GT95 parameter (i.e., the global TEG parameter, based upon 95% specificity for each of the comprising parameters), as the CR-TEG LY30 proved to be lacking in specificity as described above. The TCN-TEG (tPA-challenged TEG) was designed as a functional assay for depletion of antifibrinolytic reserves, and its LY30 was tested as a standalone parameter. For TCN-TEG, 75 ng human single chain tPA (tissue plasminogen activator) is added to each vial containing 360 ul of blood (e.g., whole blood, citrated blood, etc.) from the patient.

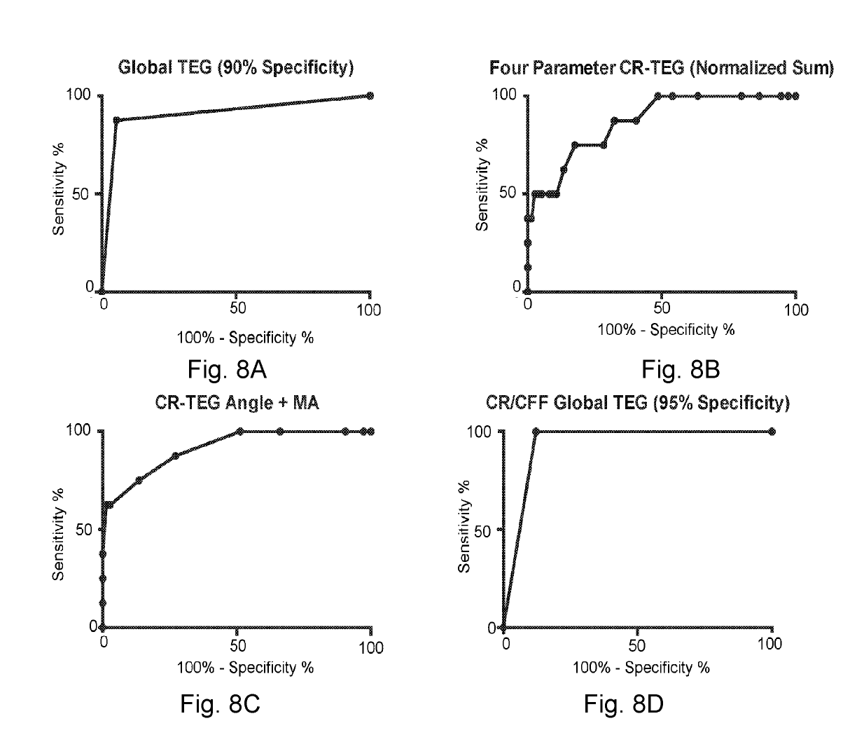

When the GT90 (global TEG parameter, based upon 9)% specificity for each of the comprising parameters) derived from the training data of Example I was applied to the prospective test data of Example II, its sensitivity was 87.5% and specificity was 94.6% for prediction of MH. Area under the curve (AUC) of the corresponding single-point ROC plot was 0.91 (p=0.0001). The FPNS yielded a ROC AUC of 0.87 (p=0.0006) with an optimized diagnostic threshold of <0.25 with a sensitivity of 75.0% and specificity of 82.4%, using new normalization scalars of −124.0, 69.0, 60.1, and −4.9 for ACT, α-angle, MA and LY30, respectively. The Angle+MA parameter had an AUC of 0.91 (p=0.0001) with an optimized threshold of <1.95 with 87.5% sensitivity and 73.0% specificity. The re-derived GT95 parameter had an AUC of 0.94 (p<0.0001) with a sensitivity of 100% and specificity of 87.8%, incorporating thresholds of >152 seconds, <61.2 degrees, and <49 mm for CRT-TEG ACT, α-angle and MA and >2.5% for CFF-TEG LY30 (See FIGS. 8A-8D). In FIG. 8A, for the global TEG parameter originally derived at 90% specificity thresholds in the retrospective group (GT90), area under the ROC curve (AUC) of the corresponding single-point ROC plot was 0.91 (p=0.0001) with sensitivity of 87.5% and specificity of 94.6% for prediction of MH. In FIG. 8B, the AUC for the four-parameter TEG normalized sum (FPNS) was 0.87 (p=0.0006) with an optimized diagnostic threshold of <0.25 yielding a sensitivity of 75.0% and specificity of 82.4%. In FIG. 8C, the AUC was 0.91 (p=0.0001) for the Angle+MA parameter with an optimized threshold of <1.95 yielding 87.5% sensitivity and 73.0% specificity. The re-derived global TEG at 95% component specificity (GT95) parameter in FIG. 8D had an AUC of 0.94 (p<0.0001) with a sensitivity of 100% and specificity of 87.8%, incorporating thresholds of >152 seconds, <61.2 degrees, and <49 mm for CRT-TEG ACT, α-angle and MA and >2.5% for CFF-TEG LY30.

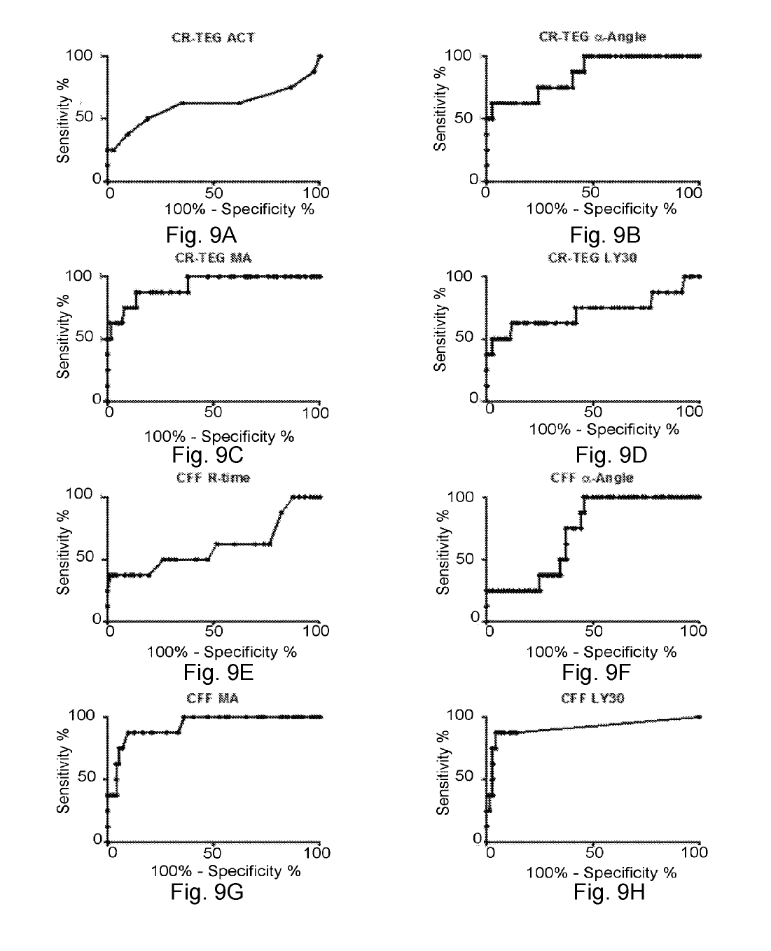

ROC curves for prediction of MH based on the four basic TEG parameters of CR-TEG and CFF-TEG in the prospective group are shown in FIGS. 9A-9H. These plots were used re-derive the scalars and threshold values for the four-parameter TEG normalized sum (FPNS) and GT95 in the prospective group, as described above. Briefly, the ROC curves for CR-TEG ACT and CFF-TEG R were not significantly different from the line of identity. AUCs for CR-TEG α-angle, MA and LY30 were 0.86, 0.92 and 0.72, respectively. AUCs for CFF-TEG α-angle, MA and LY30 were 0.72, 0.93 and 0.91, respectively. Both the CR-TEG LY30 and CFF-TEG α-angle plots were of marginal statistical significance (p=0.04).

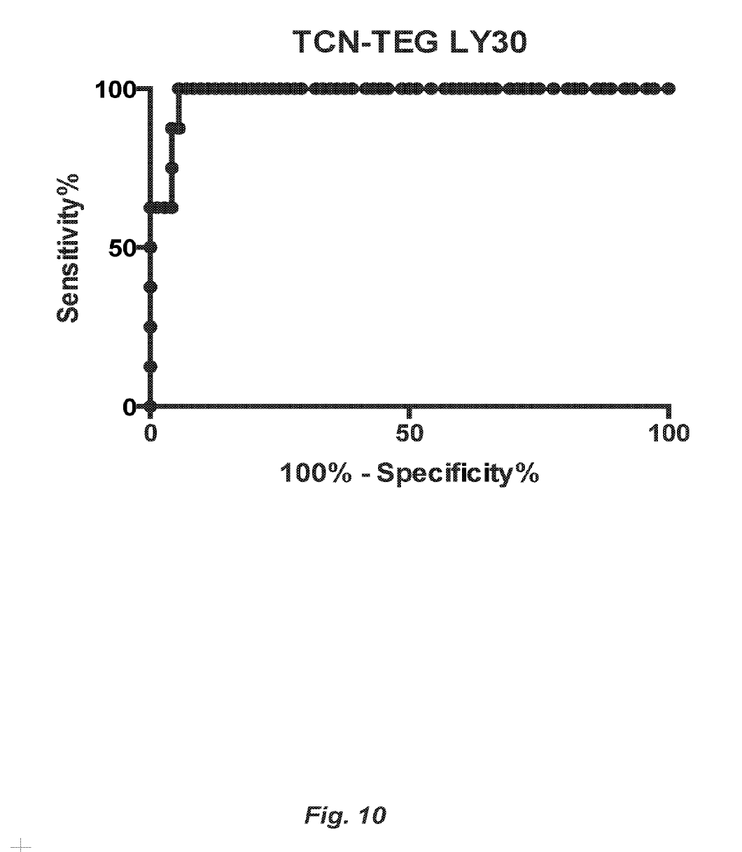

Lastly, the TCN-TEG (tPA-challenged TEG) was evaluated for predictive power with respect to MH. The LY30 in response to tPA-challenge was the only parameter extracted from this assay, as other parameters are still in a preliminary stage of development. The ROC plot of the TCN-TEG LY30 is shown in FIG. 10. AUC was 0.98 (p<0.0001), with optimized sensitivity of 100.0% and specificity of 94.4% at a threshold of LY30>54.7%.

The results provided herein, including Example I and II, demonstrate that derangements of TEG-derived coagulation parameters are powerful predictors of MH in patients suffering traumatic injury. This finding was valid both in a retrospective analysis of very high risk patients (those who triggered an MTP based upon clinician judgment) as well as a prospective analysis of a broader group of trauma activation patients whose blood sample for TEG was drawn at the earliest possible time to injury, usually in the field. The validity of this finding in the latter group is of great practical importance, as it suggests that very early TEG samples are useful for prediction of MH (and thus for guiding activation of MTPs and mobilization of other resources) even in a general trauma population where the pretest probability of MH is <10%. Coupled with recent advances in VHA technology which may allow placement of sophisticated coagulation analyzers in helicopters or ground ambulance units, these findings hold the promise of high accuracy prediction of bleeding risk and transfusion requirements before the patient even arrives at the hospital.

Trauma scoring systems such as TASH, ABC, the ROCVS criteria and the MTP trigger criteria developed at the Denver Health Medical Center (Denver, Colo., USA) were all designed to achieve this same goal using metrics available at the point of care. All of these scoring systems fell well short of the predictive value of any of the TEG-based parameters, generally having ROC plots not distinguishable from chance. Calculating these scores also requires multiple metrics (including 1 a FAST exam), which require an experienced clinician to interpret. INR and ISS likewise had no significant predictive utility for MH.

TEG, however, may have some limitations as well. TEG is, by its nature, more specific than sensitive for bleeding prediction, as TEG is insensitive to massive hemorrhage of purely mechanical (i.e. non-coagulopathic) cause. In a briskly bleeding patient who has not yet developed a coagulopathy the TEG will be normal, much as the hematocrit of a patient who is rapidly bleeding whole blood will remain normal until hemorrhagic shock is quite advanced. Moreover, individual TEG parameters each interrogate only certain aspects of the hemostatic system. TIC is a polymorphous entity, and while patients with the most severe TIC will eventually display a pan-coagulopathy affecting every stage of clot formation from enzymatic initiation to fibrinolysis, most patients display a heterogeneous mixture of more subtle and isolated disturbances of coagulation. It is precisely these patients, who are in early TIC, who must be accurately and rapidly diagnosed so that they can be treated with appropriate hemostatic resuscitation therapy. Thus, to accurately interpret the TEG, each of the major parameters which describe the tracing shape are, in some embodiments, analyzed either sequentially (as in the GT95 parameter) or each of the major parameters which describe the tracing shape are analyzed in aggregate (as in the FPNS or Angle+MA) parameters.

In some embodiments, rather than a single TEG assay, the best predictive power comes from a combined use of the CR-TEG and CFF-TEG assays, with the CR-TEG generally being more useful for early TEG parameters (α-angle and MA and to a lesser extent ACT) while CFF-TEG is more useful for the analysis of 1 the late TEG tracing features from MA to LY30. This is not surprising, as the platelet-inhibited basis of the CFF-TEG assay is more suited to detecting subtle derangements of fibrin structure and quality (e.g. factor XIII integration) as well as hyperfibrinolysis; however, the removal of platelet function masks the important contribution of platelets to clot growth which would normally be observed in the early TEG curve, as represented by R-time and α-angle. Neither of these assays extracts very good predictive power from the ACT/R-time parameters which quantitate the rate of the enzymatic initiation phase of clotting. While extreme prolongations of ACT or R are highly specific for coagulopathy, sensitivity of these parameters is extremely poor. This may be simply a function of their occurrence extremely early in the clotting process, before other aspects of the hemostatic system (e.g. fibrinogen concentration) may manifest themselves in contributing to the TEG signal. Alternatively, it may be that the very strong activators in the CR-TEG and CFF-TEG reagents may simply “wash out” a weak coagulopathy signal in this portion of the TEG tracing, much like the loss of detail in an overexposed photograph. This warrants further investigation using other TEG assays employing gentler clot activators.

A significant challenge in designing and interpreting the present study lies in properly defining the “disease positive” population, in the sense of building a two by-two contingency table. A transfusion requirement of 10 units of PRBCs in 6 hours (or death from hemorrhage prior to then) is used herein as the definition of MH. This somewhat arbitrary threshold left out several patients in prospective group who might reasonably have been judged to have MH by other standards, such as one patient who received 8 units of PRBCs, 6 of fresh frozen plasma and a resuscitative thoracotomy. It was decided, however, that very conservative, non subjective and, above all, simple criteria were necessary to avoid over-fitting the predictive model described herein to specific clinical data and losing generalizability of applicability. This simple definition for MH along with blinding of clinicians to the TEG data in the prospective analysis, eliminates many forms of bias and enhances the validity and applicability of the prognostic criteria developed herein.

Another, more fundamental, ambiguity is whether the population of predictive interest is only patients with TIC or all patients with MH, whether coagulopathic or not. From a practical standpoint, this question seems to be resolving itself. While little progress has been made on rigorously defining clinical coagulopathy much less in deriving a gold standard biochemical assay for it, increasing bodies of evidence indicate that the relationship between MH and TIC is causally cyclic, and that the one may be essentially used as proxy for the other, owing to their entanglement.

Moreover, measurable signs of coagulopathy appear to be present in nearly every patient who is severely injured enough to eventual suffer MH, so early detection of subtle coagulopathy may indeed be the best means of evaluating all patients for their risk of major bleeding. This is best illustrated by the hypersensitivity to exogenous tPA seen in the LY30 parameter of the TCN-TEG in every patient who went on to have MH (100% sensitivity), even in the very early samples collected at the scene of injury. Interestingly, the TCN-TEG flagged as positive two patients with apparently normal initial conventional TEGs who later decompensated, displaying profound coagulopathy with associated MH. These findings suggest that a latent form of TIC exists in some patients, which can be revealed by challenge with exogenous tPA.

VHA-derived parameters are the best available predictors of MH in the severely injured trauma population. Thus, VHA analysis of blood samples obtained in the field or upon arrival is probably the best basis for triggering an MTP. The best combination of commercially available TEG assays for this task is CR-TEG and CFF10 TEG. The simplest application of the parameters extracted from these assays is the GT95 parameter.

This can be implemented clinically as a simple algorithm whereby the following parameters are analyzed sequentially as they are reported by the evolving TEG tracings: CRT-TEG ACT, α-angle, and MA as well as CFF-TEG LY30. Thresholds of >152 seconds, <61.2 degrees, <49 mm and >2.5% should be applied to these parameters, respectively, and the test is scored positive if any of these four parameters is positive. If CFF is not available, the CR-TEG LY30 may be substituted, using an LY30 threshold of >3.9%. Note that the “Absolute MA” mode in the TEG 5000 software package should be selected in order to most accurately report LY30. These recommendations are intended for application to very early blood samples either drawn in the field or at the time of admission, and are fundamentally prognostic in their utility. A positive GT95 test may be useful for triggering an MTP, or raising the clinician's index of suspicion for occult bleeding or coagulopathy.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A method for identifying a patient as likely to have an onset of massive hemorrhage, comprising:
measuring at least one of a first coagulation characteristic parameter reflective of a clotting time in a sample of blood of the patient using a viscoelastic assay to obtain a first result, a second coagulation characteristic parameter reflective of clot formation in a sample of blood of the patient using the viscoelastic assay to obtain a second result; a third coagulation characteristic parameter reflective of clot strength in a sample of blood of the patient using the viscoelastic assay to obtain a third result; and a fourth coagulation characteristic parameter reflective of clot lysis in a sample of blood of the patient using the viscoelastic assay to obtain a fourth result;
wherein, a positive result for at least one of the first result, second result, third result and fourth result identifies the patient as likely to have an onset of massive hemorrhage.

2. The method of claim 1, wherein:
the first coagulation characteristic parameter is selected from the group consisting of an activated clotting time (ACT) value, a clotting time (CT) value, a reaction time (R) value, and a split point (SP) value;
the second coagulation characteristic parameter is selected from the group consisting of an α-angle value, a K value, and a CFT value;
the third coagulation characteristic parameter is selected from the group consisting of a maximum amplitude (MA) value, a maximum clot firmness (MCF) value, an A5 value, a A10 value, an A15 value, and an A20 value; and
the fourth coagulation characteristic parameter is selected from the group consisting of an LY30 value and an LI30 value.

3. The method of claim 2, wherein the first result is positive if the first result is greater than or equal to a value that is equivalent to 152 seconds when the first coagulation characteristic parameter is an ACT value as measured by a thromoboelastography viscoelastic analysis assay.

4. The method of claim 2, wherein the second result is positive if the second result is less than or equal to a value that is equivalent to 61.2 degrees when the second coagulation characteristic parameter is an α-angle value as measured by a thromoboelastography viscoelastic analysis assay.

5. The method of claim 2, wherein the third result is positive if the third result is less than or equal to a value that is equivalent to 49 mm when the third coagulation characteristic parameter is an MA value as measured by a thromoboelastography viscoelastic analysis assay.

6. The method of claim 2, wherein the fourth result is positive if the fourth result is greater than or equal to a value that is equivalent to 2.5% when the fourth coagulation characteristic parameter is an LY30 value as measured by a thromoboelastography viscoelastic analysis assay.

7. The method of claim 1, wherein the viscoelastic assay is performed using a thromboelastography analyzer system or a thromboelastometry analyzer system.

8. The method of claim 1, wherein the patient is a human patient.

9. The method of claim 1, wherein the patient is a trauma patient.

10. A method for identifying a patient as likely to have an onset of massive hemorrhage, comprising:
(a) obtaining a normalized first result by:
(i) measuring a first coagulation characteristic parameter reflective of a clotting time in a sample of blood of the patient using a viscoelastic assay to obtain a first result, and
(ii) dividing the first result by a mean of the first coagulation characteristic parameter in samples of blood of trauma patients using the viscoelastic assay to obtain the normalized first result;
(b) obtaining a normalized second result by:
(i) measuring a second coagulation characteristic parameter reflective of clot formation in a sample of blood of the patient using the viscoelastic assay to obtain a second result; and
(ii) dividing the second result by a mean of the second coagulation characteristic parameter in samples of blood of trauma patients using the viscoelastic assay to obtain the normalized second result;
(c) obtaining a normalized third result by:
(i) measuring a third coagulation characteristic parameter reflective of clot strength in a sample of blood of the patient using the viscoelastic assay to obtain a third result; and
(ii) dividing the third result by a mean of the third coagulation characteristic parameter in samples of blood of trauma patients using the viscoelastic assay to obtain the normalized third result;
(d) obtaining a normalized fourth result by:
(i) measuring a fourth coagulation characteristic parameter reflective of clot formation speed time in a sample of blood of the patient using the viscoelastic assay to obtain a fourth result; and
(ii) dividing the fourth result by a mean of the fourth coagulation characteristic parameter in samples of blood of trauma patients using the viscoelastic assay to obtain the normalized fourth result; and
(e) obtaining a value by subtracting the sum of the normalized third result and the normalized second result from the sum of the normalized first result and the normalized fourth result;
wherein a value that is greater than a threshold value established between healthy volunteers and positive control test subjects at a site of usage identifies the patient as likely to have an onset of massive hemorrhage.

11. The method of claim 10, wherein the viscoelastic assay is performed using a thromboelastography analyzer system or a thromboelastometry analyzer system.

12. The method of claim 10, wherein:
the first coagulation characteristic parameter is selected from the group consisting of an activated clotting time (ACT) value, a clotting time (CT) value, a reaction time (R) value, and a split point (SP) value;
the second coagulation characteristic parameter is selected from the group consisting of an α-angle value, a K value, and a CFT value;
the third coagulation characteristic parameter is selected from the group consisting of a maximum amplitude (MA) value, a maximum clot firmness (MCF) value, an A5 value, a A10 value, an A15 value, and an A20 value; and
the fourth coagulation characteristic parameter is selected from the group consisting of an LY30 value and an LI30 value.

13. The method of claim 10, wherein the patient is a human patient.

14. The method of claim 10, wherein the patient is a trauma patient.

15. A method for identifying a patient as likely to have an onset of massive hemorrhage, comprising:
(a) obtaining a normalized first result by:
(i) measuring a first coagulation characteristic parameter reflective of clot formation in a sample of blood of the patient using the viscoelastic assay to obtain a first result; and
(ii) dividing the second result by a mean of the first coagulation characteristic parameter in samples of blood of trauma patients using the viscoelastic assay to obtain the normalized first result;
(b) obtaining a normalized second result by:
(i) measuring a second coagulation characteristic parameter reflective of clot strength in a sample of blood of the patient using the viscoelastic assay to obtain a second result; and
(ii) dividing the second result by a mean of the second coagulation characteristic parameter in samples of blood of trauma patients using the viscoelastic assay to obtain the normalized second result; and
(c) adding the normalized first result to the normalized second result to obtain a sum, where a sum that is greater than a threshold value established between healthy volunteers and positive control test subjects at a site of usage identifies the patient as likely to have an onset of massive hemorrhage.

16. The method of claim 15, wherein the viscoelastic assay is performed using a thromboelastography analyzer system or a thromboelastometry analyzer system.

17. The method of claim 15, wherein:
the first coagulation characteristic parameter is selected from the group consisting of an α-angle value, a K value, and a CFT value; and
the second coagulation characteristic parameter is selected from the group consisting of a maximum amplitude (MA) value, a maximum clot firmness (MCF) value, an A5 value, a A10 value, an A15 value, and an A20 value.

18. The method of claim 15, wherein the patient is a human patient.

19. The method of claim 15, wherein the patient is a trauma patient.
